SEQUENCE LISTING (34 sequences)

SEQ ID NO: 1 - protein, 8 aa, synthetic construct
SYLPPLTT

SEQ ID NO: 2 - protein, 6 aa, synthetic construct
YHIEPV

SEQ ID NO: 3 - protein, 5 aa, synthetic construct
YLLVK

SEQ ID NO: 4 - protein, 7 aa, synthetic construct
SYLPPLT

SEQ ID NO: 5 - protein, 5 aa, synthetic construct
FLLVK

SEQ ID NO: 6 - protein, 5 aa, synthetic construct
WLLVK

SEQ ID NO: 7-9 - skipped (3 entries)

SEQ ID NO: 10 - protein, 4 aa, synthetic construct
YLLV

SEQ ID NO: 11 - protein, 4 aa, synthetic construct
LLVK

SEQ ID NO: 12 - protein, 4 aa, synthetic construct
YLVK

SEQ ID NO: 13 - protein, 5 aa, synthetic construct
YLLVR

SEQ ID NO: 14 - protein, 5 aa, synthetic construct
YLPPL

SEQ ID NO: 15-19 - skipped (5 entries)

SEQ ID NO: 20 - protein, 6 aa, synthetic construct
SYLPPL

SEQ ID NO: 21 - protein, 7 aa, synthetic construct
NYLLVKG

SEQ ID NO: 22 - protein, 6 aa, synthetic construct
YLLAVK

SEQ ID NO: 23 - protein, 6 aa, synthetic construct
YLLNNK

SEQ ID NO: 24 - protein, 5 aa, synthetic construct
SYLPP

SEQ ID NO: 25 - protein, 5 aa, synthetic construct
  VARIANT 1: note = Any aromatic amino acid
XLLVK

SEQ ID NO: 26 - protein, 17 aa, synthetic construct
YETLSYLPPLTTDQLAR

SEQ ID NO: 27 - skipped

SEQ ID NO: 28 - protein, 16 aa, synthetic construct
YKGRCYHIEPVAGEEN

SEQ ID NO: 29-30 - skipped (2 entries)

SEQ ID NO: 31 - protein, 7 aa, synthetic construct
  VARIANT 1: note = Y, F, W, or H
  VARIANT 3: note = Y, F, W, Q, or L
XLXEIAR

SEQ ID NO: 32 - protein, 5 aa, synthetic construct
VYLPR

SEQ ID NO: 33 - protein, 4 aa, synthetic construct
YLPR

SEQ ID NO: 34 - protein, 5 aa, synthetic construct
VLQRF

(19) United States
(12) Patent Application Publication
(10) Pub. No.: US 2024/0166686 A1
(43) Pub. Date: May 23, 2024

(54) THERAPEUTIC PEPTIDES

(71) Applicants: Kyoto University, Kyoto-shi (JP); Kazusa DNA Research Institute, Kisarazu-shi (JP)

(72) Inventors: Kousaku OHINATA, Kyoto-shi (JP); Saeko KIMURA, Kyoto-shi (JP); Hideyuki SUZUKI, Kisarazu-shi (JP)

(73) Assignees: Kyoto University, Kyoto-shi (JP); Kazusa DNA Research Institute, Kisarazu-shi (JP)

(21) Appl. No.: 18/320,512

(22) Filed: May 19, 2023

Related U.S. Application Data
(63) Continuation of application No. 16/490,376, filed on Aug. 30, 2019, now abandoned.

(30) Foreign Application Priority Data
Mar. 4, 2017 (JP) 2017-041181

(51) Int. Cl. C07K 5/087; A23L 33/105; A23L 33/18; A23L 33/185; C07K 5/10; C07K 7/06; C07K 7/08; A61K 38/00

(52) U.S. Cl. CPC C07K 5/0812; A23L 33/105; A23L 33/18; A23L 33/185; C07K 5/10; C07K 7/06; C07K 7/08; A23V 2002/00; A61K 38/00

(57) ABSTRACT

The present invention provides peptides, salts thereof, peptide conjugates, and salts thereof having an anxiolytic-like effect, for example a peptide comprising any amino acid sequence selected from (i) an amino acid sequence SYLPPLTT (SEQ ID NO: 1), (ii) an amino acid sequence YHIEPV (SEQ ID NO: 2), (iii) an amino acid sequence YLLVK (SEQ ID NO: 3), (iv) an amino acid sequence SYLPPLT (SEQ ID NO: 4), (v) an amino acid sequence FLLVK (SEQ ID) NO. 5), and (vi) an amino acid sequence WLLVK (SEQ ID NO: 6).

### TECHNICAL FIELD

This application claims the priority benefit of Japanese application no. 2017-041181 filed on Mar. 4, 2017, the contents of which are incorporated herein in their entireties by reference thereto.

### 1. Technical Field

The present invention relates to peptides and peptide conjugates. The present invention also relates to pharmaceutical compositions and foods comprising the peptides and peptide conjugates.

### SEQUENCE LISTING

This application contains a Sequence Listing which has been submitted electronically in XML format. The Sequence Listing XML is incorporated herein by reference. Said XML file, created on Jan. 31, 2024, is named DGS-003USC1_SL.xml and is 24 kilobytes in size.

### BACKGROUND ART

### 2. Background

Japan, which now finds itself at the forefront of a super-aged society, is expected to achieve active and healthy aging. So-called soft foods focused on a decline in chewing and swallowing functions have been developed so far as foods adapted to elderly people. However, there is substantially no food focused on a decline in the functions of the nervous system, such as physiological anorexia of aging, decrease in motivation and increase in mental stress.

Such mental stress might increase the risk of developing life style-related diseases.

It will be required to develop next-generation functional foods for caregiving capable of slowing, halting or reversing the decline in nerve functions.
Non Patent Literature 1 states that spinach-derived Rubiscolin-6 has an anxiolytic effect.

### CITATION LIST

### Non Patent Literature

[Non Patent Literature 1] Hirata H, Sonoda S, Agui S, Yoshida M, Ohinata K, Yoshikawa M. Peptides. 2007 October; 28 (10): 1998-2003.

### SUMMARY OF INVENTION

### Technical Problem

An object of the present invention is to provide peptides having an anxiolytic-like effect, and a pharmaceutical and a food comprising such peptides.

### Solution to Problem

### 3. Summary

The present inventor has aimed at developing functional ingredients mitigating mental stress and consequently identified peptides that show anxiolytic-like effects or antidepressant-like effects. The present invention has been completed through further studies based on these findings.

Specifically, the present invention encompasses the following aspects.

In one aspect, the disclosure provides peptides, salts thereof, peptide conjugates, and salts thereof (sometimes collectively referred to herein as “compounds”) derived from pepsin and pepsin+pancreatin digests of a RubisCo protein, for example peptides having an amino acid sequence of any of SEQ ID NO: 1 to SEQ ID NO:6. It should be understood that when an embodiment described herein refers to a “peptide,” the embodiment encompasses the peptide per se as well as salts of the peptide even though the embodiment may not explicitly recite the expression “or salt thereof” or similar, unless required otherwise by context. Likewise, disclosure relating to a peptide encompasses conjugates of the peptide and salts of the conjugate unless required otherwise by context.

In one aspect, the disclosure provides a peptide that is 3 to 20 amino acids in length and has an amino acid sequence comprising at least 3 consecutive amino acids from the N-terminal or C-terminal end of the amino acid sequence X₁LX₂X₃VK (SEQ ID NO:8), where X₁ is a hydrophobic amino acid, X₂ and X₃ are each independently selected from any amino acid, preferably selected from hydrophobic amino acids. As used herein, “hydrophobic amino acid” means an amino acid with a hydrophobic side chain, such as A, I, L, M, V, F, W, and Y. In some embodiments, the hydrophobic amino acid is a naturally occurring amino acid. In some embodiments, one, two, or all three of X₁, X₂, and X₃ are aromatic amino acids (e.g., Y, F, or W). In some embodiments, the peptide comprises 4 or 5 consecutive amino acids from the N-terminal or C-terminal end of the amino acid sequence of SEQ ID NO:8. Accordingly, the peptide can comprise X₁, L, X₂ or X₃ at its N-terminal position. The peptide may also comprise other amino acids N-terminal to X₁, L, X₂ or X₃. Preferably, the N-terminal amino acid is a hydrophobic amino acid. Such SEQ ID NO:8-based peptides can be 3, 4, 5, 6, 7, 8, 9, 10, 11, 12, 13, 14, 15, 16, 17, 18, 19, or 20 amino acids in length, or may have a length ranging between any pair of the foregoing embodiments, e.g., 3 to 10 amino acids in length, 4 to 9 amino acids in length, 4 to 18 amino acids in length, 5 to 12 amino acids in length, 6 to 15 amino acids in length, 6 to 10 amino acids in length, and so on and so forth.

In another aspect, the disclosure provides a peptide 5 to 20 amino acids in length and comprises the amino acid sequence SYLPP (SEQ ID NO:24) at the N-terminal end of the peptide. Such SEQ ID NO:24-based peptides can be 5, 6, 7, 8, 9, 10, 11, 12, 13, 14, 15, 16, 17, 18, 19, or 20 amino acids in length, or may have a length ranging between any pair of the foregoing embodiments, e.g., 5 to 10 amino acids in length, 5 to 9 amino acids in length, 6 to 18 amino acids in length, 5 to 12 amino acids in length, 6 to 15 amino acids in length, 6 to 10 amino acids in length, and so on and so forth.

In another aspect, the disclosure provides a peptide that is 6 to 20 amino acids in length and comprises the amino acid sequence YHIEPV (SEQ ID NO:2). Such SEQ ID NO:2-based peptides can 6, 7, 8, 9, 10, 11, 12, 13, 14, 15, 16, 17, 18, 19, or 20 amino acids in length, or may have a length ranging between any pair of the foregoing embodiments, e.g., 6 to 10 amino acids in length, 6 to 9 amino acids in length, 6 to 18 amino acids in length, 6 to 12 amino acids in length, 7 to 15 amino acids in length, 7 to 10 amino acids in length, and so on and so forth.

In another aspect, the disclosure provides pharmaceutical compositions and food products comprising a peptide or peptide conjugate of the disclosure.

In another aspect, the disclosure provides methods for treating or preventing a mood disorder, an anxiety disorder, or a disorder of diminished motivation using the peptides, peptide conjugates, pharmaceutical compositions, and food products described herein.

In one aspect, the present disclosure provides the following embodiments labeled Item 1 to Item 3:

Item1. A peptide comprising at least 3 consecutive amino acids from the amino acid sequence XLLVK (SEQ ID NO:25) (in SEQ ID NO:25, X is an aromatic amino acid). As used herein, “aromatic amino acid” means an amino acid with an aromatic side chain (e.g., F, W, and Y).

Item2. The peptide according to Item1, wherein X is Y (tyrosine), F (phenylalanine) or W (tryptophan).

Item3. A peptide comprising any amino acid sequence selected from the following amino acid sequence or the following amino acid sequence in which 1 to 3 amino acid(s) are deleted, replaced and/or added,
(i) an amino acid sequence SYLPPLTT (SEQ ID NO: 1),
(ii) an amino acid sequence YHIEPV (SEQ ID NO: 2),
(iii) an amino acid sequence YLLVK (SEQ ID NO: 3),
(iv) an amino acid sequence SYLPPLT (SEQ ID NO: 4),
(v) an amino acid sequence FLLVK (SEQ ID NO: 5), and
(vi) an amino acid sequence WLLVK (SEQ ID NO: 6).

In another aspect, the present disclosure provides the following embodiments labeled Item 1 to Item 26:

Item 1. A peptide comprising any amino acid sequence selected from
(i) an amino acid sequence SYLPPLTT (SEQ ID NO: 1),
(ii) an amino acid sequence YHIEPV (SEQ ID NO: 2),
(iii) an amino acid sequence YLLVK (SEQ ID NO: 3),
(iv) an amino acid sequence SYLPPLT (SEQ ID NO: 4),
(v) an amino acid sequence FLLVK (SEQ ID NO: 5), and
(vi) an amino acid sequence WLLVK (SEQ ID NO: 6).

Item 2. A pharmaceutical composition comprising a peptide according to item 1 as an active ingredient.

Item 3. A pharmaceutical composition comprising a peptide according to item 1 and a pharmacologically acceptable diluent, carrier, or excipient.

Item 4. The pharmaceutical composition according to item 2, wherein the pharmaceutical composition is a therapeutic agent for anxiety disorder.

Item 5. The pharmaceutical composition according to item 2, wherein the pharmaceutical composition is a therapeutic agent for depression.

Item 6. The pharmaceutical composition according to item 2, wherein the pharmaceutical composition is a therapeutic agent for adjustment disorder.

Item 7. The pharmaceutical composition according to item 2, wherein the pharmaceutical composition is a therapeutic agent for bipolar disorder.

Item 8. The pharmaceutical composition according to item 2, wherein the pharmaceutical composition is a therapeutic agent for disorder of the will.

Item 9. The pharmaceutical composition according to item 2, wherein the pharmaceutical composition is a therapeutic agent for apathy.

Item 10. The pharmaceutical composition according to item 2, wherein the pharmaceutical composition is a therapeutic agent for abulia.

Item 11. The pharmaceutical composition according to item 2, wherein the pharmaceutical composition is a therapeutic agent for akinetic mutism.

Item 12. An oral drug comprising a pharmaceutical composition according to any one of item 2 to 11.

Item 13. A food comprising a peptide according to item 1.

Item 14. A food which is supplemented with a peptide according to item 1.

Item 15. The food according to item 13 or 14 for the slowing, halting or reversing an anxiety state.

Item 16. A composition comprising RubisCo protein treated with pepsin or pancreatin.

Item 17. A food comprising a composition according to item 16.

Item 18. A food which is supplemented with a composition according to item 16.

Item 19. A method for recovering or treating anxiety disorder or a symptom based on anxiety, comprising the step of administering a peptide according to item 1 to a patient with anxiety disorder or the symptom based on anxiety or a potential sufferer thereof.

Item 20. The peptide according to item 1 for use in the recovery or treatment of anxiety disorder or a symptom based on anxiety.

Item 21. Use of a peptide according to item 1 for producing a pharmaceutical or a food for the recovery or treatment of anxiety disorder or a symptom based on anxiety.

Item 22. A green plant for use in the recovery or treatment of anxiety disorder or a symptom based on anxiety.

Item 23. A method for recovering or treating decrease in motivation, depression, depressive mood disorders or a symptom based on thereof, comprising the step of administering the peptide according to Item 1 to a patient with decrease in motivation, depression, depressive mood disorders or a symptom based on thereof or a potential sufferer thereof.

Item 24. The peptide according to Item 1 for use in the recovery or treatment of depression, depressive mood disorders or a symptom based on thereof.

Item 25. Use of the peptide according to Item 1 for producing a pharmaceutical or a food for the recovery or treatment of depression, depressive mood disorders or a symptom based on thereof.

Item 26. A leaf of green plant for use in the recovery or treatment of depression, depressive mood disorders or a symptom based on thereof.

### Advantageous Effects of Invention

A pharmaceutical composition and a food comprising the peptide of the present invention as an active ingredient can have a high anxiolytic-like effect with low adverse reaction and are suitable for long-term use, and/or have an antidepressant-like effect (hereinafter antidepressant-like effect includes antidepressant effect). Also, the pharmaceutical composition and the food of the present invention are suitable for oral administration.

Natural short-chain peptides may be ingested as foods. It can be expected that diseases are prevented in individuals with an anxiety state by the ingestion of these peptides as foods.

One aspect of the peptide of the present invention is an enzymatic digest of a chloroplast protein and is therefore free from adverse reaction problems. Furthermore, the chloroplast RubisCo protein is abundant in green plants and can therefore be produced at low cost.

### 4. Brief Description of the Drawings

### BRIEF DESCRIPTION OF DRAWINGS

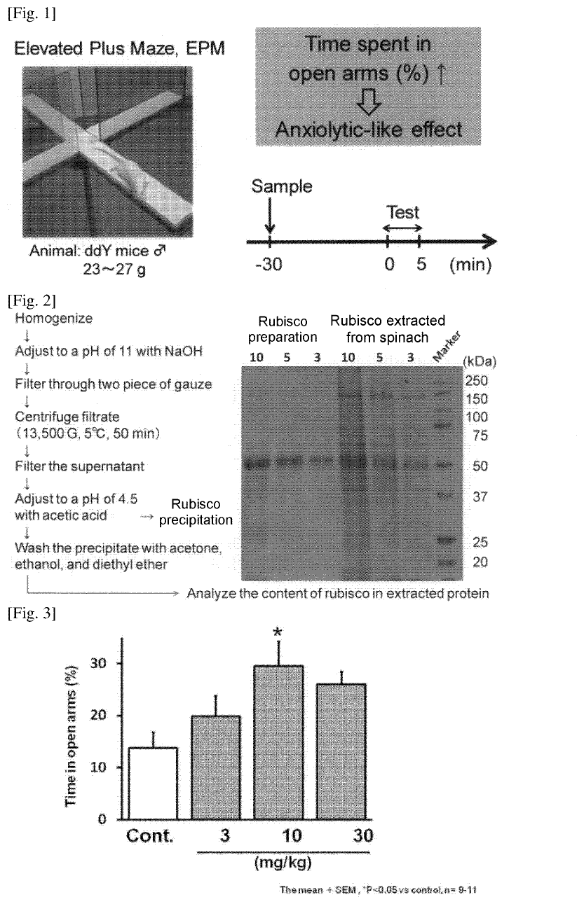

FIG. 1 shows a testing method for an elevated plus-maze test (EPM). The test was conducted 30 minutes after oral administration of a sample, and the evaluation was conducted based on the 5-minute test time. 23 to 27 g male ddY mice were used as animals to be studied. Cumulative time in open arms was recorded, and the percentage (%) of the time in open arms was used as an index for an anxiolytic-like effect.

FIG. 2 shows a process for the extraction of RubisCo protein from spinach and result of SDS-PAGE.

FIG. 3 shows results of the elevated plus-maze test of Example 1.

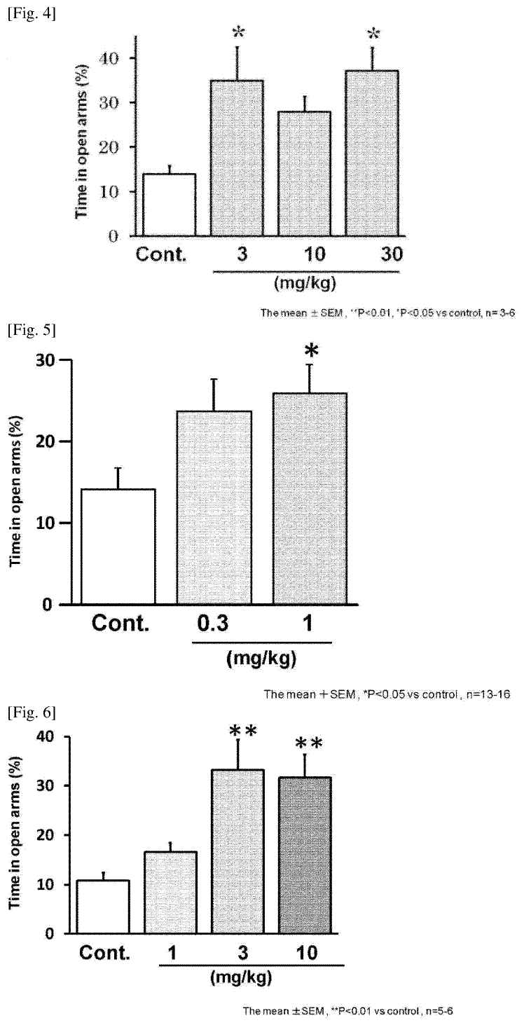

FIG. 4 shows results of the elevated plus-maze test of Example 2.

FIG. 5 shows results of the elevated plus-maze test of Example 3.

FIG. 6 shows results of the elevated plus-maze test of Example 4.

FIG. 7 shows results of the elevated plus-maze test of Example 5.

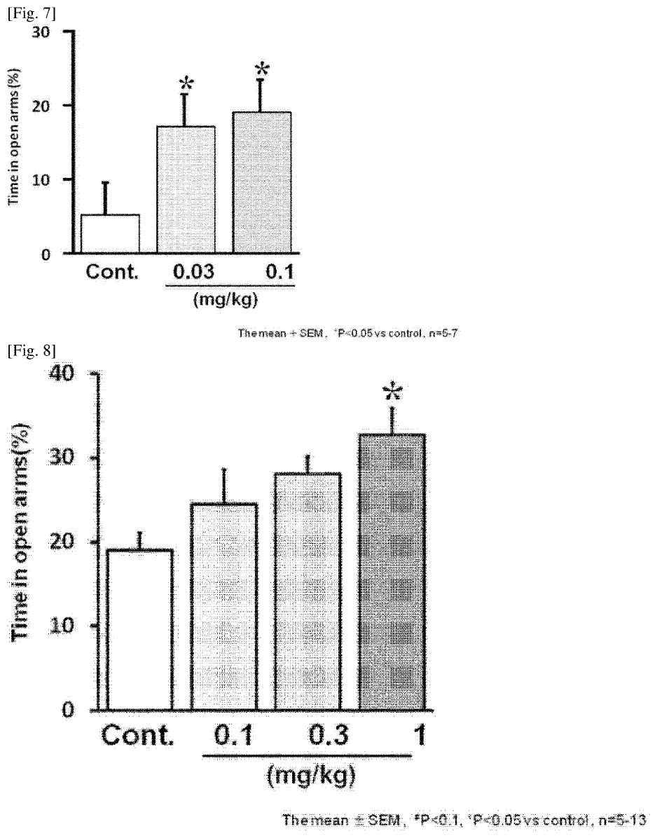

FIG. 8 shows results of the elevated plus-maze test of Example 6.

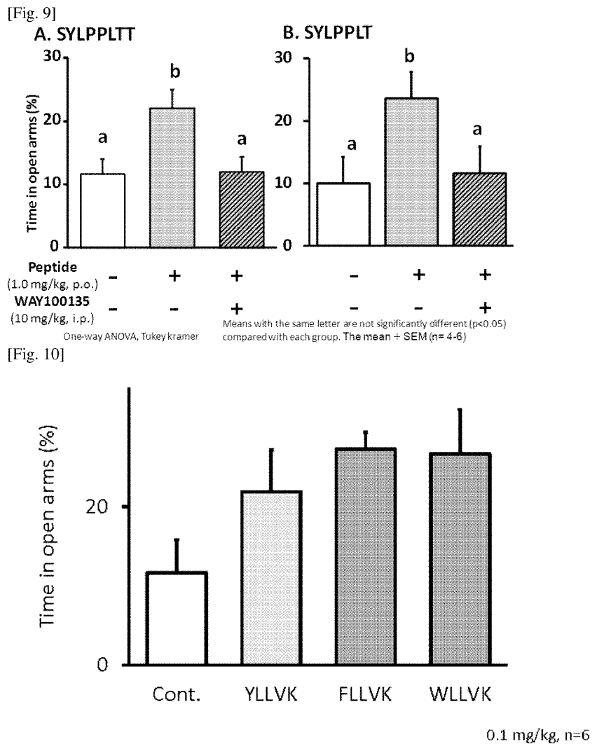

FIG. 9 shows results of studying the mechanism of action of an enzymatic digest using an antagonist as described in Example 7.

FIG. 10 shows results of the elevated plus-maze test of Example 8.

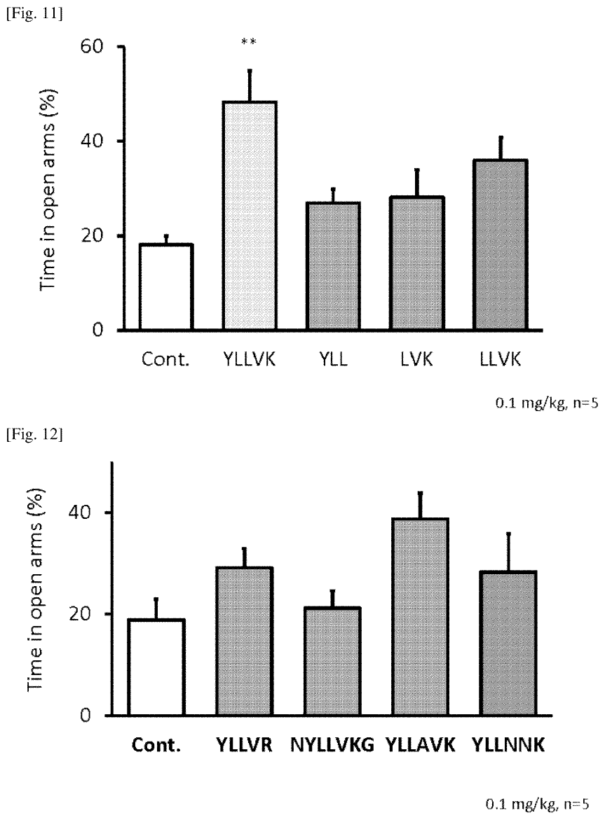

FIG. 11 shows results of the elevated plus-maze test of Example 9.

FIG. 12 shows results of the elevated plus-maze test of Example 10.

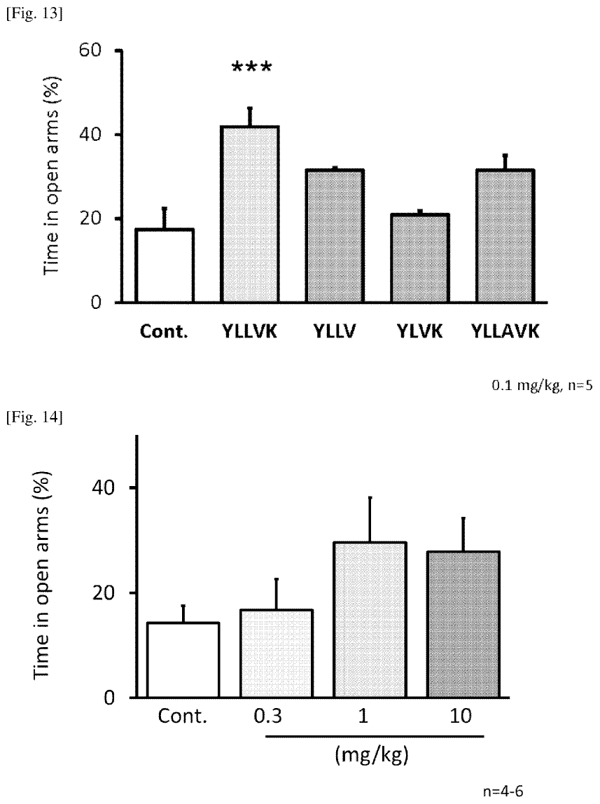

FIG. 13 shows results of the elevated plus-maze test of Example 11.

FIG. 14 shows results of the elevated plus-maze test of Example 12.

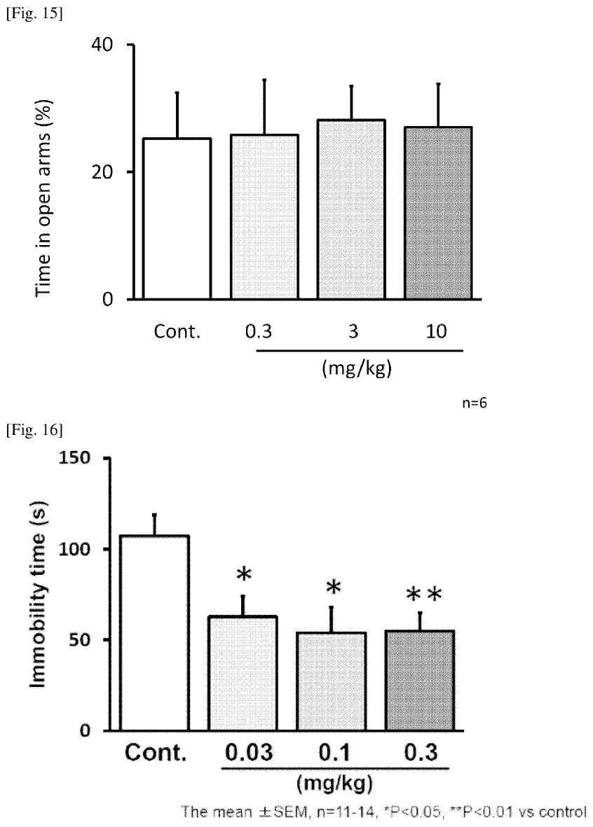

FIG. 15 shows results of the elevated plus-maze test of Example 13.

FIG. 16 shows results of the tail suspension test of Example 14.

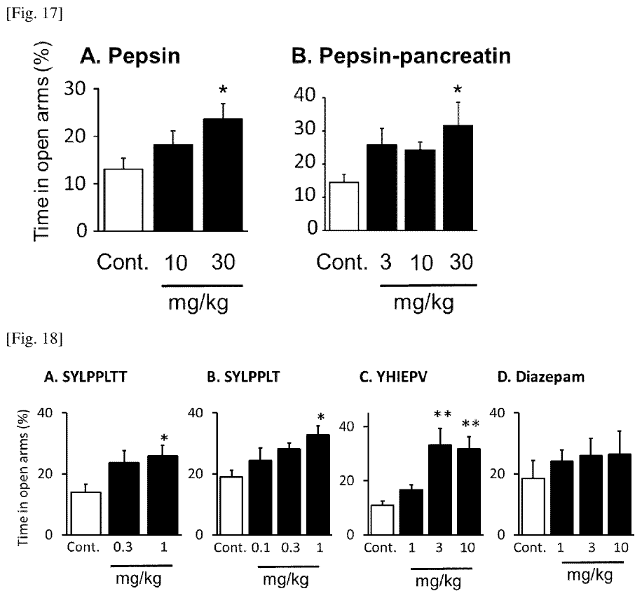

FIG. 17 shows results of the elevated plus-maze test of Example 15.

FIG. 18 shows results of the elevated plus-maze test of Example 16.

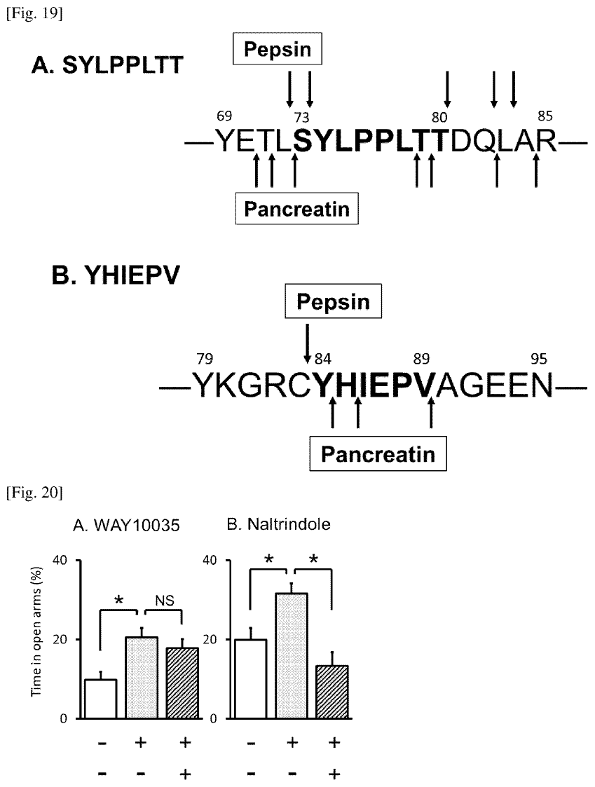

FIG. 19 shows cleavage sites in spinach RubisCo small subunit (A) and large subunit (B) by gastrointestinal proteases.

FIG. 20 shows results of the elevated plus-maze test of Example 18.

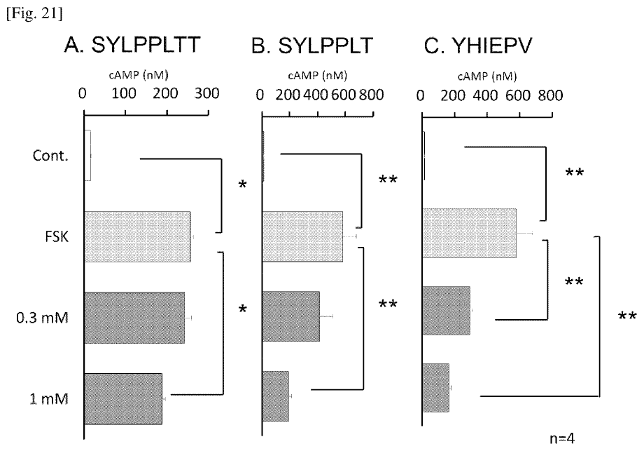

FIG. 21 shows results of the intracellular cAMP elevation assay of Example 19.

### DESCRIPTION OF EMBODIMENTS

### 5. Detailed Description

### 5.1 Peptides

In one aspect, the disclosure provides peptides and salts thereof derived from pepsin and pepsin+pancreatin digests of a RubisCo protein, for example peptides having an amino acid sequence of any of SEQ ID NO: 1 to SEQ ID NO:6. The peptide having the amino acid sequence YLLVK (SEQ ID NO:3), one of the preferred peptides of the disclosure, was identified in pepsin and pepsin+pancratin digests of spinach Rusico protein. In subsequent digests, however, the peptide YLLVK (SEQ ID NO:3) was not detected. Nevertheless, when the peptide was characterized as described in the Examples, it was found to have anxiolytic-like effect. Exemplary peptides based on the amino acid sequences of SEQ ID NOS: 1-6, including peptides based on the amino acid sequences of SEQ ID NOS: 8, 24 and 28, are described herein.

### 5.1.1 Peptides Based on SEQ ID NOS:1-6

In certain aspects, the peptide of the present invention is a peptide comprising any amino acid sequence selected from the following amino acid sequence or the following amino acid sequence in which 1 to 3 amino acid(s) are deleted, replaced and/or added; (i) an amino acid sequence SYLPPLTT (SEQ ID NO: 1), (ii) an amino acid sequence YHIEPV (SEQ ID NO: 2), (iii) an amino acid sequence YLLVK (SEQ ID NO: 3), (iv) an amino acid sequence SYLPPLT (SEQ ID NO: 4), (v) an amino acid sequence FLLVK (SEQ ID NO: 5), and (vi) an amino acid sequence WLLVK (SEQ ID NO: 6).

In some embodiments, the peptide has an amino acid sequence corresponding to any one of SEQ ID NO: 1-6, but which has 1 deletion compared to the amino acid sequence of any one of SEQ ID NO:1-6. In some embodiments, the peptide has an amino acid sequence corresponding to any one of SEQ ID NO: 1-6, but which has 2 deletions compared to the amino acid sequence of any one of SEQ ID NO: 1-6. In some embodiments, the peptide has an amino acid sequence corresponding to any one of SEQ ID NO:1-6, but which has 3 deletions compared to the amino acid sequence of any one of SEQ ID NO:1-6.

In some embodiments, the peptide has an amino acid sequence corresponding to any one of SEQ ID NO:1-6, but which has 1 amino acid substitution compared to the amino acid sequence of any one of SEQ ID NO: 1-6. In some embodiments, the peptide has an amino acid sequence corresponding to any one of SEQ ID NO:1-6, but which has 2 amino acid substitutions compared to the amino acid sequence of any one of SEQ ID NO: 1-6. In some embodiments, the peptide has an amino acid sequence corresponding to any one of SEQ ID NO:1-6, but which has 3 amino acid substitutions compared to the amino acid sequence of any one of SEQ ID NO:1-6.

In some embodiments, the peptide has an amino acid sequence corresponding to any one of SEQ ID NO:1-6, but which has 1 amino acid added compared to the amino acid sequence of any one of SEQ ID NO: 1-6. In some embodiments, the peptide has an amino acid sequence corresponding to any one of SEQ ID NO: 1-6, but which has 2 amino acids added compared to the amino acid sequence of any one of SEQ ID NO: 1-6. In some embodiments, the peptide has an amino acid sequence corresponding to any one of SEQ ID NO:1-6, but which has 3 amino acids added compared to the amino acid sequence of any one of SEQ ID NO:1-6.

In another aspect, the peptide of the present invention is a peptide comprising any amino acid sequence selected from (i) an amino acid sequence SYLPPLTT (SEQ ID NO: 1), (ii) an amino acid sequence YHIEPV (SEQ ID NO: 2), (iii) an amino acid sequence YLLVK (SEQ ID NO: 3), (iv) an amino acid sequence SYLPPLT (SEQ ID NO: 4), (v) an amino acid sequence FLLVK (SEQ ID NO: 5), and (vi) an amino acid sequence WLLVK (SEQ ID NO: 6).

The peptides of disclosure based on the amino acid sequences of SEQ ID NOS:1-6, may or may not include amino acids other than the amino acid sequences of SEQ ID NOS: 1-6, respectively. Accordingly, in various embodiments:
(1) the peptides of the disclosure comprising the amino acid sequences of SEQ ID NOS: 3, 5 and 6 may be up to 6, up to 7, up to 8, up to 9, up to 10, up to 12, up to 15, up to 18, up to 20 amino acids or up to 25 amino acids in length;
(2) the peptides of the disclosure comprising the amino acid sequence of SEQ ID NO: 2 may be up to 6, up to 7, up to 8, up to 9, up to 10, up to 12, up to 15, up to 18, up to 20 amino acids or up to 25 amino acids in length;
(3) the peptides of the disclosure comprising the amino acid sequence of SEQ ID NO:4 may be up to 7, up to 8, up to 9, up to 10, up to 12, up to 15, up to 18, up to 20 amino acids or up to 25 amino acids in length; and
(4) the peptides of the disclosure based on the amino acid sequence of SEQ ID NO: 1 may be up to 8, up to 9, up to 10, up to 12, up to 15, up to 18, up to 20 amino acids or up to 25 amino acids in length.

Amino acid residue(s) can be added to the N-terminal and/or C-terminal side (preferably, C-terminal side) of a peptide of the present invention as long as the resulting peptide comprises an amino acid sequence described above. The number of the amino acid residue(s) to be added is not limited and can be approximately 20 amino acid residues, preferably approximately 10 amino acid residues, more preferably approximately 5 amino acid residues, further preferably 4, 3, 2, or 1 amino acid residue(s).

One or more (e.g., 2 or 3), preferably 1 amino acid residue(s) in the amino acid sequence can also be substituted as long as the effect of the present invention is not impaired. One or more (e.g., 2 or 3), preferably 1 amino acid residue(s) can also be inserted into the amino acid sequence as long as the effect of the present invention is not impaired. One or more (e.g., 2 or 3), preferably 1 amino acid residue(s) in the amino acid sequence can also be deleted as long as the effect of the present invention is not impaired.

In some embodiments, no amino acid residue is added to the N-terminal side of SYLPPLTT (SEQ ID NO: 1), SYLPPLT (SEQ ID NO: 4), SYLPPL (SEQ ID NO: 20).

One preferred form of the peptide of the present invention includes a peptide consisting of (i) an 8-residue amino acid sequence of the amino acid sequence SYLPPLTT (SEQ ID NO: 1), (ii) a 6-residue amino acid sequence of YHIEPV (SEQ ID NO: 2), (iii) a 5-residue amino acid sequence of YLLVK (SEQ ID NO: 3), (iv) a 7-residue amino acid sequence of SYLPPLT (SEQ ID NO: 4), (v) a 5-residue amino acid sequence of FLLVK (SEQ ID NO: 5), or (vi) a 5-residue amino acid sequence of WLLVK (SEQ ID NO: 6).

### 5.1.2 SEQ ID NO:8-Based Peptides

In one aspect, the disclosure provides a peptide that is 3 to 20 amino acids in length and has an amino acid sequence comprising at least 3 (e.g., 3, 4, 5, or 6) consecutive amino acids from the N-terminal or C-terminal end of the amino acid sequence X₁LX₂X₃VK (SEQ ID NO:8), where X₁ is a hydrophobic amino acid, X₂ and X₃ are each independently selected from any amino acid, preferably selected from hydrophobic amino acids. In some embodiments, the peptide has a hydrophobic N-terminal amino acid.

In some embodiments, the peptide is 3 to 15 amino acids in length. In other embodiments, the peptide is 3 to 10 amino acids in length. In other embodiments, the peptide is 4 to 20 amino acids in length. In other embodiments, the peptide is 4 to 15 amino acids in length. In other embodiments, the peptide is 4 to 10 amino acids in length. In other embodiments, the peptide is 5 to 20 amino acids in length. In other embodiments, the peptide is 5 to 15 amino acids in length. In other embodiments, the peptide is 5 to 10 amino acids in length.

In some embodiments, the peptide is 3 amino acids in length. In other embodiments, the peptide is 4 amino acids in length. In other embodiments, the peptide is 5 amino acids in length. In other embodiments, the peptide is 6 amino acids in length. In other embodiments, the peptide is 7 amino acids in length. In other embodiments, the peptide is 8 amino acids in length. In other embodiments, the peptide is 9 amino acids in length. In other embodiments, the peptide is 10 amino acids in length. In other embodiments, the peptide is 11 amino acids in length. In other embodiments, the peptide is 12 amino acids in length. In other embodiments, the peptide is 13 amino acids in length. In other embodiments, the peptide is 14 amino acids in length. In other embodiments, the peptide is 15 amino acids in length. In other embodiments, the peptide is 16 amino acids in length. In other embodiments, the peptide is 17 amino acids in length. In other embodiments, the peptide is 18 amino acids in length. In other embodiments, the peptide is 19 amino acids in length. In other embodiments, the peptide is 20 amino acids in length.

X₁ can be any hydrophobic amino acid, for example, alanine (A), isoleucine (I), leucine (L), methionine (M), valine (V), phenylalanine (F), tryptophan (W), or tyrosine (W). In some embodiments, X₁ is an aromatic amino acid selected from F, W, and Y. In some embodiments, X₁ is F. In other embodiments, X₁ is W. In other embodiments, X₁ is Y.

X₂ and X₃ are preferably hydrophobic amino acids such as A, I, L, M, V, F, or W. X₂ and X₃ can be the same or different. In some embodiments, X₂ and X₃ are the same. In other embodiments, X₂ and X₃ are different. In some embodiments, X₂ and/or X₃ is selected from L, I, V, and A. In some embodiments, X₂ is L. In some embodiments, X₂ is I. In some embodiments, X₂ is V. In some embodiments, X₂ is A. In some embodiments, X₃ is L. In some embodiments, X₃ is I. In some embodiments, X₃ is V. In some embodiments, X₃ is A.

In some embodiments, the N-terminal amino acid of the peptide is X₁. In some embodiments, the two N-terminal amino acids of the peptide are X₁L, for example, YL, FL, or WL.

In some embodiments, the C-terminal amino acid of the peptide is K. In some embodiments, the two C-terminal amino acids of the peptide are VK.

In some embodiments, the first the two N-terminal amino acids of the peptide are X₁L, for example, YL, FL, or WL, and the two C-terminal amino acids are VK. For example, the peptide can have the amino acid sequence YLLVK (SEQ ID NO:3), FLLVK (SEQ ID NO:5), or WLLVK (SEQ ID NO:6).

### 5.1.3 SEQ ID NO:24-Based Peptides

In another aspect, the disclosure provides a peptide 5 to 20 amino acids in length and comprising the amino acid sequence SYLPP (SEQ ID NO:24) at the N-terminal end of the peptide. In some embodiments, the peptide comprises or consists of the amino acid sequence SYLPP (SEQ ID NO:24). In some embodiments, the peptide comprises or consists of the amino acid sequence SYLPPL (SEQ ID NO:20). In some embodiments, the peptide comprises or consists of the amino acid sequence SYLPPLT (SEQ ID NO:4). In some embodiments, the peptide comprises or consists of the amino acid sequence SYLPPLTT (SEQ ID NO:1).

In some embodiments, the peptide is 5 to 15 amino acids in length. In other embodiments, the peptide is 5 to 10 amino acids in length.

In some embodiments, the peptide is 5 amino acids in length. In some embodiments, the peptide is 6 amino acids in length. In some embodiments, the peptide is 7 amino acids in length. In some embodiments, the peptide is 8 amino acids in length. In some embodiments, the peptide is 9 amino acids in length. In some embodiments, the peptide is 10 amino acids in length. In other embodiments, the peptide is 6 amino acids in length. In other embodiments, the peptide is 7 amino acids in length. In other embodiments, the peptide is 8 amino acids in length. In other embodiments, the peptide is 9 amino acids in length. In other embodiments, the peptide is 10 amino acids in length. In other embodiments, the peptide is 11 amino acids in length. In other embodiments, the peptide is 12 amino acids in length. In other embodiments, the peptide is 13 amino acids in length. In other embodiments, the peptide is 14 amino acids in length. In other embodiments, the peptide is 15 amino acids in length. In other embodiments, the peptide is 16 amino acids in length. In other embodiments, the peptide is 17 amino acids in length. In other embodiments, the peptide is 18 amino acids in length. In other embodiments, the peptide is 19 amino acids in length. In other embodiments, the peptide is 20 amino acids in length. The peptide can comprise, for example, the amino acid sequence SYLPP (SEQ ID NO:24), and one or more additional amino acids C-terminal to the SYLPP (SEQ ID NO:24) sequence in a RubisCo protein, for example as shown in SEQ ID NO:26.

### 5.1.4 Additional SEQ ID NO:2-Based Peptides

In another aspect, the disclosure provides a peptide that is 6 to 20 amino acids in length and comprising the amino acid sequence YHIEPV (SEQ ID NO:2). In some embodiments, the peptide is 6 to 15 amino acids in length. In other embodiments, the peptide is 6 to 10 amino acids in length.

In some embodiments, the peptide is 6 amino acids in length. In some embodiments, the peptide is 7 amino acids in length. In some embodiments, the peptide is 8 amino acids in length. In some embodiments, the peptide is 9 amino acids in length. In some embodiments, the peptide is 10 amino acids in length. In other embodiments, the peptide is 11 amino acids in length. In other embodiments, the peptide is 12 amino acids in length. In other embodiments, the peptide is 13 amino acids in length. In other embodiments, the peptide is 14 amino acids in length. In other embodiments, the peptide is 15 amino acids in length. In other embodiments, the peptide is 16 amino acids in length. In other embodiments, the peptide is 17 amino acids in length. In other embodiments, the peptide is 18 amino acids in length. In other embodiments, the peptide is 19 amino acids in length. In other embodiments, the peptide is 20 amino acids in length. The peptide can comprise, for example, the amino acid sequence YHIEPV (SEQ ID NO:2), and one or more additional amino acids N-terminal and/or C-terminal to the YHIEPV (SEQ ID NO:2) sequence in a RubisCo protein, for example as shown in SEQ ID NO:28.

### 5.1.5 SEQ ID NO:25-Based Peptides

In another aspect, the peptide of the present invention is a peptide comprising at least 3 (e.g., 3, 4, or 5) consecutive amino acids from the amino acid sequence XLLVK (SEQ ID NO:25), where X is an aromatic amino acid.

The amino acid residues of the peptides based on the amino acid sequences of any one of SEQ ID NOS: 1-6, 8, 24, and 25 as described above can include both naturally- and/or non-naturally-occurring amino acid residue(s), unless otherwise specified. The natural amino acid includes amino acid residues which constitute protein, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and other amino acid residues such as selenocysteine, N-formylmethionine, pyrrolysine, and pyroglutamine. Exemplary non-natural amino acids include, but are not limited to azetidinecarboxylic acid, 2-aminoadipic acid, 3-aminoadipic acid, β-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline

Any of L-amino acids, D-amino acids, and DL-amino acids (including any of racemates and amino acids having an excess of any one of enantiomers as long as they are mixtures of D- and L-amino acids) can be used as the amino acids constituting the peptide. A peptide consisting of only L-amino acids or only D-amino acids is preferred.

When the peptide used in the present invention contains two or more asymmetric carbon atoms, this peptide may be in any form of each enantiomer or diastereomer or a mixture of enantiomers or diastereomers at any ratio. Enantiomers and diastereomers can be separated using a column which is commonly used. Any known method can be used for separation, such as a method using an optically active column, a method which involves performing optical resolution in the form of a derivative having an introduced optically active group, and then removing the optically active group, or a method which involves forming an optically active salt with an acid or a base, followed by optical resolution.

The peptide of the present invention can be a salt (acid-addition salt or basic salt). Examples of the acid-addition salt include: inorganic salts such as hydrochloride, sulfate, nitrate, phosphate, hydrobromide, and perchlorate; and salts of organic acids such as citric acid, succinic acid, maleic acid, fumaric acid, malic acid, tartaric acid, p-toluenesulfonic acid, benzenesulfonic acid, methanesulfonic acid, and trifluoroacetic acid. Examples of the basic salt include: salts of alkali metals such as sodium, potassium, and lithium; and salts of alkaline earth metals such as calcium and magnesium. Exemplary bases that can be used to make a base addition salt include sodium hydroxide, potassium hydroxide, and bases of alkali metals, such as lithium hydroxide, calcium hydroxide. Additional acids and bases that can be used to make pharmaceutically acceptable salts are described in Stahl and Wermuth, eds., 2008, Handbook of Pharmaceutical Salts: Properties, Selection and Use, Verlag Helvetica Chimica Acta, Zurich, Switzerland, the contents of which are incorporated herein by reference in their entireties.

The peptides of the present invention can be a solvate. Examples of the solvate include solvates with water (for hydrates), methanol, ethanol, isopropanol, acetic acid, tetrahydrofuran, acetone, dimethylformamide, dimethyl sulfoxide, dimethylacetamide, acetamide, ethylene glycol, propylene glycol, dimethoxyethane, or the like.

In one aspect, some peptides of the present invention can be obtained by the hydrolysis of ribulose 1,5-bisphosphate carboxylase/oxygenase (RubisCo) protein with pepsin or with pepsin and pancreatin.

Thus, the present invention encompasses a composition comprising RubisCo protein treated with pepsin or pancreatin, or pepsin+pancreatin.

RubisCo protein is a protein involved in the carbon dioxide fixation by green leaves. This protein is abundant in plants and is considered as the most abundant protein on earth. The green plants containing RubisCo protein include plants having edible green leaf portions, such as kale, young barley leaves, young wheat leaves, ashitaba, young mulberry leaves, spinach, mulukhiya, cabbage, and tea. Spinach-derived RubisCo protein is preferred for obtaining the peptide having the amino acid sequence SYLPPLTT (SEQ ID NO: 1), the amino acid sequence YHIEPV (SEQ ID NO: 2), or the amino acid sequence SYLPPLT (SEQ ID NO: 4). Tea-derived RubisCo protein is preferred for obtaining the peptide having the amino acid sequence YHIEPV (SEQ ID NO: 2) or the amino acid sequence SYLPPLT (SEQ ID NO: 4). The having the amino acid sequence YLLVK (SEQ ID NO: 3) is preferably obtained by chemical synthesis or by recombinant expression as described below.

Pepsin (EC.3.4.23.1-3) is one type of known protease that works in the stomachs of animals. Pepsin can be used as a food additive in Japan. A commercially available product of reagent grade pepsin, food additive grade pepsin, or the like can be used.

Pancreatin is a mixture of enzymes secreted from the pancreas and includes lipase, amylase, protease (trypsin, chymotrypsin, etc.), and the like. Pancreatin can be used as a food additive in Japan. A commercially available product of reagent grade pancreatin, food additive grade pancreatin, or the like can be used.

The substrate to be hydrolyzed with pepsin or with pepsin and pancreatin is not particularly limited as long as the substrate contains RubisCo protein. Examples thereof include green plants themselves, squeezes of green plants (so-called green juices), and purified RubisCo protein.

RubisCo protein is conveniently extracted from spinach which permits its extraction as a soluble protein.

The hydrolysis with pepsin or with pepsin and pancreatin is performed under conditions that allow a peptide of the present invention to be obtained. The reaction temperature can be appropriately selected from 30 to 70° C., 30 to 40° C., 40 to 70° C., 50 to 65° C., etc. The reaction time can be appropriately selected from approximately 30 minutes to 48 hours, approximately 1 to 10 hours, approximately 2 to 8 hours, etc. The pH at which the reaction is performed can be appropriately selected from approximately pH 1.5 to 3.5, preferably approximately pH 2 to 3, for the pepsin and from approximately pH 6.5 to 8.5, preferably approximately pH 7 to 8, for the pancreatin.

In the case of performing the hydrolysis using both pepsin and pancreatin, it is preferred to separately perform the respective hydrolysis reactions, because the enzymes differ in optimum pH. In this case, the order of the hydrolysis with pepsin and the hydrolysis with pancreatin is not limited, and either of the reactions can be performed first.

If necessary, each enzyme is deactivated by heating to a temperature that permits the deactivation of the enzyme (e.g., heating at a temperature exceeding 80° C. for approximately 5 to 60 minutes).

The hydrolysis reaction product can be used directly for a pharmaceutical application or food, or the active ingredient peptide can be separated by purification and used for a pharmaceutical application or food.

Alternatively, a peptide of the present invention may be obtained by any known peptide synthesis method (e.g., as described in Benoiton, N., 2006, Chemistry of Peptide Synthesis, CRC Press, Boca Raton, FL; Howl, J., ed., 2005, Peptide Synthesis and Applications, Humana Press, Totowa, NJ; Chan and White, eds., 2000, Fmoc Solid Phase Synthesis: A Practical Approach, Oxford University Press, Oxford, UK). Specifically, in a liquid-phase method or a solid-phase method which is a method commonly used in peptide synthesis, a starting material having a reactive carboxyl group and a starting material having a reactive amino group can be condensed by a common method of peptide synthesis, for example, a method using an active ester such as HBTU or a method using a coupling agent such as carbodiimide. When the resulting condensate has a protective group, the protective group can be removed to produce the peptide.

A functional group that should not be involved in the reaction in this reaction process is protected with a protective group. Examples of the protective group for an amino group include benzyloxycarbonyl (CBZ), t-butyloxycarbonyl (Boc), and 9-fluorenylmethyloxycarbonyl (Fmoc). Examples of the protective agent for a carboxyl group include groups capable of forming alkyl ester, benzyl ester, etc. In the solid-phase method, the C-terminal carboxyl group is bonded to a carrier such as chlorotrityl resin, chloromethyl resin, oxymethyl resin, or p-alkoxybenzyl alcohol resin. The condensation reaction is carried out in the presence of a condensing agent such as carbodiimide or using active ester of a N-protected amino acid or active ester of a peptide.

After the completion of the condensation reaction, the protective group is removed. In the solid-phase method, the bond between the C terminus of the peptide and the resin is further cleaved. In addition, the peptide of the present invention is purified according to a common method. Examples thereof include ion-exchange chromatography, reverse-phase liquid chromatography, and affinity chromatography. The synthesis of the synthesized peptide is analyzed by a protein sequencer which reads an amino acid sequence from the C terminus by the Edman degradation technique, GC-MS, or the like.

The peptides of the present invention may be synthesized by an enzymatic method (see WO2003/010307).

The peptides of the present invention may be obtained from microorganisms or cultured cells which are genetic manipulated to produce a peptide of the present invention such as inserting a gene encoding such peptide therein or may be obtained by in vitro translation.

### 5.2 Peptide Conjugates

The disclosure provides peptide conjugates and salts thereof that comprise a peptide moiety and a conjugate moiety. Attachment of a conjugate moiety to a peptide can provide, for example, improved water solubility, improved stability, and reduced clearance as compared to the non-conjugated peptide (Hamley, 2014, Biomacromolecules 15:1543-1559). Thus, peptide conjugates can in some instances be more suitable as therapeutic agents compared to their unconjugated counterparts. The peptide moiety can comprise any peptide described herein, for example any peptide described in Section 3 or Section 5.1. It should be understood that when an embodiment described herein refers to a “peptide conjugate,” the embodiment encompasses the peptide conjugate per se as well as salts of the peptide conjugate even though the embodiment may not explicitly recite the expression “or salt thereof” or similar, unless required otherwise by context. Exemplary salts include the acid addition and base addition salts described in Section 5.1.

The peptide conjugates comprise one or more conjugate moieties (e.g., 1, 2, 3, 4, or 5 conjugate moieties) attached to the peptide moiety. The conjugate moiety or moieties can be attached to an N-terminal amino acid, a C-terminal amino acid, an amino acid that is neither an N-terminal amino acid or a C-terminal amino acid, or a combination thereof. For example, a peptide conjugate can comprise one conjugate moiety, preferably which is either attached to the N-terminal amino acid of the peptide moiety or attached to the C-terminal amino acid of the peptide moiety. As another example, a peptide conjugate can comprise two conjugate moieties, one of which is preferably attached to the N-terminal amino acid of the peptide moiety and the other of which is preferably attached to the C-terminal amino acid of the peptide moiety.

In embodiments in which the peptide conjugate comprises multiple conjugate moieties, each of the conjugate moieties can be the same, some of the conjugate moieties can be the same and others can be different, or all of the conjugate moieties can be different. For example, a peptide conjugate having two conjugate moieties can have two of the same conjugate moiety. Alternatively, a peptide conjugate having two conjugate moieties can have two different conjugate moieties. As another example, a peptide conjugate having three conjugate moieties can have three of the same conjugate moiety, three different conjugate moieties, or two of the same conjugate moiety and one different conjugate moiety.

A conjugate moiety can be attached to a peptide moiety, for example, at one of the peptide moiety's amino acid side chains, its backbone, its N-terminal amino group, or its C-terminal carboxylic acid group. For example, a conjugate moiety can be attached to an amino acid side chain to form a chemically modified amino acid, such as methionine sulfoxide, methionine sulfone, S-(carboxymethyl)-cysteine, S-(carboxymethyl)-cysteine sulfoxide and S-(carboxymethyl)-cysteine sulfone. Other side chain modifications include acylation of lysine ε-amino groups, N-alkylation of arginine, histidine, or lysine, and alkylation of glutamic or aspartic carboxylic acid groups. Conjugate moieties can be attached to the peptide backbone, for example to a nitrogen atom in the backbone (e.g., a methyl conjugate moiety can be introduced into a peptide conjugate's backbone by using an N-methyl amino acid to synthesize the peptide). Conjugate moieties can be attached to the N-terminal amino group of the peptide moiety to provide, for example, an N-terminus having an N-lower alkyl, N-di-lower alkyl, or N-acyl modifications. Conjugate moieties can be attached to the C-terminal carboxy group to provide, for example, a peptide conjugate having an amide, a lower alkyl amide, a dialkyl amide, or a lower alkyl ester at the C-terminus of the conjugate. A lower alkyl refers to a C1-C4 alkyl.

Exemplary conjugate moieties that can be used in the peptide conjugates include polymers, amine groups (e.g., amino (—NH₂), alkyl amino and dialkyl amino), acyls groups (e.g., formyl or acetyl), alkyl groups (e.g., C1-C4 alkyl), phosphate groups, lipids and sugars.

In some embodiments, at least one, more than one, or all of the conjugate moieties in the peptide conjugate comprise(s) a polymer. Exemplary polymers that can be used as conjugate moieties include polyethylene glycol, polyvinyl pyrrolidone, polylactic-co-glycolic acid, N-(2-hydroxypropyl) methacrylamide copolymer, polyglutamic acid, and polysaccharides. In some embodiments, at least one, more than one, or all of the conjugate moieties in the peptide conjugate comprise(s) a polyethylene glycol. In some embodiments, at least one, more than one, or all of the conjugate moieties in the peptide conjugate comprise(s) polyvinyl pyrrolidone. In some embodiments, at least one, more than one, or all of the conjugate moieties in the peptide conjugate comprise(s) polylactic-co-glycolic acid. In some embodiments, at least one, more than one, or all of the conjugate moieties in the peptide conjugate comprise(s) N-(2-hydroxypropyl) methacrylamide copolymer. In some embodiments, at least one, more than one, or all of the conjugate moieties in the peptide conjugate comprise(s) polyglutamic acid. In some embodiments, at least one, more than one, or all of the conjugate moieties in the peptide conjugate comprise(s) comprises a polysaccharide.

In some embodiments, at least one, more than one, or all of the conjugate moieties in the peptide conjugate comprise(s) an amine group. Exemplary amine groups include amino (—NH2), alkyl amino, and dialkyl amino groups. The alkyl groups can be, for example, a C1-C4 alkyl. In some embodiments, at least one, more than one, or all of the conjugate moieties in the peptide conjugate comprise(s) an amino group. In some embodiments, at least one, more than one, or all of the conjugate moieties in the peptide conjugate comprise(s) an alkyl amino group. In some embodiments, at least one, more than one, or all of the conjugate moieties in the peptide conjugate comprise(s) a dialkyl amino group.

In some embodiments, at least one, more than one, or all of the conjugate moieties in the peptide conjugate comprise(s) an acyl group. Exemplary acyl groups include formyl groups and acetyl groups. In some embodiments, at least one, more than one, or all of the conjugate moieties in the peptide conjugate comprise(s) a formyl group. In some embodiments, at least one, more than one, or all of the conjugate moieties in the peptide conjugate comprise(s) an acetyl group.

In some embodiments, at least one, more than one, or all of the conjugate moieties in the peptide conjugate comprise(s) an alkyl group. In exemplary embodiments, the alkyl group is a lower alkyl group, such as methyl or ethyl. In some embodiments, at least one, more than one, or all of the conjugate moieties in the peptide conjugate comprise(s) a methyl group. In some embodiments, at least one, more than one, or all of the conjugate moieties in the peptide conjugate comprise(s) an ethyl group.

In some embodiments, at least one, more than one, or all of the conjugate moieties in the peptide conjugate comprise(s) a phosphate group, for example attached to the side chain of a serine, threonine, or tyrosine.

In some embodiments, at least one, more than one, or all of the conjugate moieties in the peptide conjugate comprise(s) a lipid.

In some embodiments, at least one, more than one, or all of the conjugate moieties in the peptide conjugate comprise(s) a sugar.

Processes for attaching conjugate moieties to peptide moieties are known in the art and can be used to obtain the peptide conjugates described herein (e.g., as described in Basle et al., 2010, Chemistry & Biology 17:213-227; Benoiton, N., 2006, Chemistry of Peptide Synthesis, CRC Press, Boca Raton, FL; Ernst and Leumann, eds., 1995, Modern Synthetic Methods, Verlag Helvetica Chimica Acta, Basel, Switzerland; Hamley, 2014, Biomacromolecules 15:1543-1559; Lundblad, R., 1995, Techniques in Protein Modification, CRC Press, Boca Raton, FL). Custom synthesis of peptide conjugates is also commercially available from numerous vendors (e.g., ABI Scientific (Sterling, VA); AnaSpec (Freemont CA); Pepscan, (Lelystad, Netherlands), Neo Scientific (Cambridge, MA); Sigma-Aldrich (St. Louis, MO), variously offering, for example, peptides having N-terminal conjugate moieties such as an acetyl group, a formyl group, a fatty acid, and alkyl amino groups, peptides having C-terminal conjugate moieties such as an amido group, alkyl amino groups, and alkyl groups, peptides conjugated to fatty acids, peptides conjugated to polyethylene glycol, and peptides having a phosphate conjugate moiety (e.g., comprising phosphoserine, phosphothreonine, or phosphotyrosine)).

### 5.3 Uses of the Compounds of the Disclosure

Peptides of the present invention have anxiolytic-like effects and can be used for the treatment or recovery of an anxiety disorder or a symptom based on anxiety (e.g., anxiety and a psychological and/or physical symptom associated therewith). Peptides and peptide conjugates of the present invention can be used as an active ingredient in an anti-anxiety agent, e.g., a pharmaceutical composition as described herein.

The anxiety disorder encompasses phobia, generalized anxiety disorder, panic disorder, and substance-induced anxiety disorder. An individual that is not diagnosed with an anxiety disorder but has an anxiety state caused by stress, etc. (potential sufferer of anxiety disorder) is also included in a subject for the recovery. The treatment according to the present invention includes the procedures, alleviation and recovery of a symptom and/or the complete or partial inhibition of the progression of a disease.

The anxiolytic-like effect of a peptide or peptide conjugate can be evaluated by an elevated plus-maze test which has been developed as an anxiety-related behavior evaluation method for screening for anti-anxiety agents and is widely used (FIG. 1). Specifically, a candidate substance is orally or intraperitoneally administered to each mouse. 30 minutes later, the mouse is placed in an elevated plus-maze. The strength of the anxiolytic-like effect can be evaluated by using the number of entries into open arms and change in residence time in the open arms as indexes.

As demonstrated in Examples mentioned later, and without being bound by theory, some peptides of the present invention are believed to act via the activation of a 5-HT_{1A} receptor and can therefore be expected to also have an antidepressant-like effect. The peptides of the present invention can also be used in the treatment or recovery of depression or depressive mood disorder, or a state (symptom) based thereon.

Antidepressant-like effect can be evaluated by a tail suspension test. The tail suspension test is an experimental method used to screen potential antidepressant drugs (Can et al., 2012, J Vis Exp., 59:e3769). The amount of time that a mouse is immobile (i.e. is not displaying escape behavior) during the six minutes is measured to provide an immobility time. The administration of anti-depressant drugs such as imipramine reduces immobility time. Therefore, if a reduction of immobility time is observed when testing a test substance, it can be concluded that the test substance has anti-depressant properties. Immobility is considered a despair state, and, therefore, a reduction of immobility time also indicates an increase in motivation (i.e., that the test substance has motivation-increasing properties).

The peptides and peptide conjugates of the present invention can also be used as an active ingredient in a therapeutic agent (e.g., a pharmaceutical composition as described herein) for treating adjustment disorder, bipolar disorder, a disorder of the will (also referred to as a disorder of diminished motivation), apathy, abulia, or akinetic mutism on the basis of their anxiolytic-like effects.

The peptides and peptide conjugates of the present invention can be provided as a pharmaceutical composition or a food (also referred to herein as a “food composition”).

The administration route of the peptide or peptide conjugate of the present invention or a product containing the peptide or peptide conjugate is not particularly limited, and oral administration, parenteral administration (e.g., intramuscular or intravenous administration), intrarectal administration, or the like can be adopted. Among them, oral administration is preferred from the viewpoint of being highly effective.

The dose of the peptide or peptide conjugate of the present invention can vary depending on the type of the compound, an administration method, the state or age of a recipient, etc. and is commonly 0.01 mg/kg to 500 mg/kg, preferably 0.05 mg/kg to 100 mg/kg, more preferably 0.1 to 30 mg/kg, per day in an adult. The peptide or peptide conjugate (active ingredient) of the present invention can be administered in the form of a pharmaceutical composition prepared by mixing with a carrier for formulations. A substance that is commonly used in the field of formulations and does not react with the peptide of the present invention can be used as the carrier for formulations.

The peptide or peptide conjugate of the present invention can be used in itself as a pharmaceutical or a food. The peptide or peptide conjugate of the present invention can be prepared, either alone or with an appropriate nontoxic carrier, diluent or excipient for oral ingestion, into a formation for foods or pharmaceuticals such as a tablet (plain tablet, sugar-coated tablet, foaming tablet, film-coated tablet, chewable tablet, etc.), a capsule including any of hard capsules and soft capsules, a troche, a powder, fine granules, granules, a solution, a suspension, an emulsion, a paste, a cream, an injection (including blends with infusions such as amino acid infusions and electrolytic infusions), or a sustained-release formulation such as enteric-coated tablet, capsule, or granules. The pharmaceutical compositions of the disclosure can be formulated according to techniques known in the art (e.g., as described in Allen et al., eds., 2012, Remington: The Science and Practice of Pharmacy, 22nd Edition, Pharmaceutical Press, London, UK).

In one aspect, the present invention provides a pharmaceutical composition comprising a peptide or peptide conjugate of the present invention and a pharmacologically acceptable diluent, carrier, or excipient. In another aspect, the present invention provides a food containing a peptide or peptide conjugate of the present invention (e.g., a food which can contain a peptide of the present invention by addition).

The content amount of the peptide or peptide conjugate of the present invention in the pharmaceutical or the food can be appropriately selected and is generally in the range of 0.01 to 100% by weight (e.g., 1% to 99%, 1% to 90%, 5% to 80%, 10% to 75%, or 15% to 50% by weight of the pharmaceutical composition, or any weight percent range bound by any two of the foregoing values).

Specifically, examples of substances such as the carrier for formulations or the carrier, diluent or excipient for oral ingestion that can be added to the pharmaceutical or the food include lactose, glucose, mannitol, dextrin, cyclodextrin, starch, sucrose, magnesium aluminometasilicate, synthetic aluminum silicate, carboxymethylcellulose sodium, hydroxypropyl starch, carboxymethylcellulose calcium, ion-exchange resin, methylcellulose, gelatin, gum arabic, hydroxypropylcellulose, hydroxypropylmethylcellulose, polyvinylpyrrolidone, polyvinyl alcohol, light silicic anhydride, magnesium stearate, talc, tragacanth, bentonite, Veegum, titanium oxide, sorbitan fatty acid ester, sodium lauryl sulfate, glycerin, fatty acid glycerin ester, purified lanoline, glycerogelatin, polysorbate, macrogol, plant oil, wax, liquid paraffin, white petroleum Jelly (e.g., Vaseline(trademark)), fluorocarbon, nonionic surfactants, propylene glycol, and water.

Examples of dosage forms include tablets, capsules, granules, powders, syrups, suspensions, suppositories, ointments, creams, gels, patches, inhalants, and injections. These formulations can be prepared according to common methods. Liquid formulations can be in a form to be dissolved or suspended in water or other appropriate solvents when used. Tablets or the granules can be coated by well-known methods. The injections can prepared by dissolving a peptide or peptide conjugate of the present invention in water. If necessary, the injections can be prepared by dissolving the peptide or peptide conjugate of the present invention in physiological saline or a glucose solution and can be supplemented with a buffer or a preservative.

These formulations can contain a peptide or peptide conjugate of the present invention at a proportion of 0.01% to 100% by weight, preferably 1 to 90% by weight (e.g., 1% to 80%, 1% to 70%, 1% to 60%, 1% to 50%, 1% to 40%, 1% to 30%, 1% to 20%, 1% to 10%, 5% to 10%, 10% to 20%, 20% to 40%, 40% to 60%, 60% to 80%, or any range bounded by any two of the foregoing values). These formulations can also contain other components valuable for treatment.

In order to produce solid formulations for oral administration, the active ingredient(s) can be mixed with excipient components, for example, lactose, starch, crystalline cellulose, calcium lactate, and silicic anhydride to prepare powders, or further supplemented, if necessary, with a binder (saccharose, hydroxypropylcellulose, polyvinylpyrrolidone, etc.) or a disintegrant (carboxymethylcellulose, carboxymethylcellulose calcium, etc.) and wet- or dry-granulated to prepare granules. In order to produce tablets, these powders or granules can be compressed, either directly or after addition of a lubricant such as magnesium stearate or talc. These granules or tablets may be coated with an enteric coating base such as hydroxypropylmethylcellulose phthalate or a methacrylic acid-methyl methacrylate polymer to prepare enteric coated formulations, or coated with ethylcellulose, carnauba wax, hydrogenated oil, or the like to prepare sustained-release formulations. In order to produce capsules, the powders or the granules can be filled into hard capsule shells, or the active ingredient can be coated, either directly or after dissolution in glycerin, polyethylene glycol, sesame oil, olive oil, or the like, with a gelatin film to prepare soft capsules.

In order to produce liquid formulations for oral administration, the active ingredient and a sweetener such as saccharose, sorbitol, or glycerin can be dissolved in water and supplemented with a clear syrup and further with essential oil, ethanol, or the like to prepare elixirs, or can be supplemented with gum arabic, tragacanth, polysorbate 80, carboxymethylcellulose sodium, or the like to prepare emulsions or suspensions. These liquid formulations can be supplemented, if desired, with a corrigent, a colorant, a preservative, etc.

The food comprising a peptide or peptide conjugate of the present invention can be produced, for example, by adding the peptide or peptide conjugate of the present invention into a known food. Examples of specific forms of the food that can be produced by the addition of the peptide according to the present invention can include drinks (coffee, cocoa, juices, soft drinks, mineral drinks, tea drinks, green tea, black tea, oolong tea, milk beverages, lactic acid bacteria beverages, yogurt drinks, carbonated beverages, other nonalcoholic beverages, alcohol beverages, etc.), confectionery (hard candies, gums, gummy candies, jellies, puddings, mousses, cakes, candies, cookies, crackers, biscuits, chocolates, ices (ice creams, ice candies, sherbets, ice shavings, etc.), etc.), Furikake toppings, dressings, seasonings, processed meat foods (hamburger patty, meat loaf, meatball, Tsukune (grilled chicken meatball), etc.), processed fish foods (Kamaboko (steamed fish paste), Chikuwa (fish sausage), etc.), retort processed foods, and jelly-like foods (jellies, agars, jelly-like drinks, etc.). The food supplemented with the peptide or peptide conjugate according to the present invention can be prepared by a method known per se.

Examples of the food comprising the peptide or peptide conjugate of the present invention also include foods prepared from green plants as raw materials, such as powdered green tea, green juices, and vegetable juices. The food comprising the peptide or peptide conjugate of the present invention can be produced, for example, by a method for producing a food (powdered green tea, green juice, vegetable juice, etc.) from a green plant as a raw material, further comprising the step of hydrolysis with pepsin or hydrolysis with pepsin and pancreatin. In another aspect, the food comprising the peptide or peptide conjugate of the present invention can also be produced by a method for producing a food (powdered green tea, green juice, vegetable juice, etc.) from a green plant as a raw material, further comprising the step of forming the peptide of the present invention by freeze drying, acid and/or alkali treatment, etc.

The food comprising the peptide or peptide conjugate of the present invention can be a so-called health food, food with function claims, food for specified health use, dietary supplement (e.g., in the form of a tablet, a capsule, a softgel, a gelcap, a liquid, or a powder, and optionally including one or more ingredients selected from vitamins, minerals, herbs or other botanicals, amino acids, proteins, fiber, fatty acids, and combinations thereof), supplement, food for the sick, combined food for the sick (Ministry of Health, Labour and Welfare, Japan, one type of food for special dietary uses) or food for elderly people (Ministry of Health, Labour and Welfare, Japan, one type of food for special dietary uses).

The hydrolysis of the RubisCo protein with pepsin or with pepsin and pancreatin is considered to also occur in the digestive tract. From such a viewpoint, the present invention encompasses use of a green plant (e.g., kale, young barley leaves, young wheat leaves, ashitaba, young mulberry leaves, spinach, mulukhiya, cabbage, and tea) itself in the recovery or treatment of anxiety disorder or a symptom based on anxiety. The green plant is preferably spinach or tea, more preferably spinach.

In some aspects, the disclosure provides methods of treating a subject with a peptide, peptide conjugate, pharmaceutical composition, or food product of the disclosure.

The disclosure provides a method for treating a subject suffering from an anxiety disorder comprising administering to the subject a compound of the disclosure in an amount effective to treat the subject. The disclosure also provides a method for treating or preventing an anxiety disorder comprising administering an amount of a food product comprising a compound of the disclosure to a subject prone to or suffering from the anxiety disorder. In some embodiments, the subject is prone to suffer from an anxiety disorder. In other embodiments, the subject is suffering from an anxiety disorder.

The disclosure provides a method for treating a subject suffering from a disorder of diminished motivation comprising administering to the subject a compound of the disclosure in an amount effective to treat the subject. The disclosure also provides a method for treating or preventing a disorder of diminished motivation comprising administering an amount of a food product comprising a compound of the disclosure to a subject prone to or suffering from a disorder of diminished motivation. In some embodiments, the subject is prone to suffer from a disorder of diminished motivation. In other embodiments, the subject is suffering from a disorder of diminished motivation.

In some embodiments of the methods described in the preceding paragraph, the disorder of diminished motivation comprises apathy. In other embodiments of the methods described in the preceding paragraph, the disorder of diminished motivation comprises abulia. In other embodiments of the methods described in the preceding paragraph, the disorder of diminished motivation comprises akinetic mutism.

The disclosure provides a method for treating a subject suffering from a mood disorder comprising administering to the subject a compound of the disclosure or a pharmaceutical composition comprising a compound of the disclosure in an amount effective to treat the subject. The disclosure also provides a method for treating or preventing a mood disorder comprising administering an amount of a food product comprising a compound of the disclosure to a subject prone to or suffering from a mood disorder. In some embodiments, the subject is prone to suffer from a mood disorder. In other embodiments, the subject is suffering from a mood disorder.

In some embodiments of the methods described in the preceding paragraph, the mood disorder comprises depression. In some embodiments of the methods described in the preceding paragraph, the mood disorder comprises bipolar disorder. In some embodiments of the methods described in the preceding paragraph, the mood disorder comprises adjustment disorder.

The subjects of the methods described herein are preferably mammals, e.g., humans or domestic pets (e.g., cat, dog). Subjects can be of any age, but are preferably adults (e.g., a human subject who is 18 years old or more, 25 years old or more, 35 years old or more, 45 years old or more, 55 years old or more, etc.). In some embodiments, the subject is elderly (e.g., a human subject who is 65 years old or more, 70 years old or more, 75 years old or more, or 80 years old or more).

Appropriate daily dosages of the compounds of the disclosure can be based upon the body weight of the subject, as described above (e.g., in a dose ranging from 0.01 mg/kg to 500 mg/kg). Alternatively, the compounds can be administered at a fixed dose, for example, ranging from 0.1 mg to 50 g per day (e.g., 0.1 mg to 10 g, 0.1 mg to 3 g, 0.1 mg to 100 mg, 0.1 mg to 1 mg, 0.3 mg to 3 g, or 0.3 mg to 100 mg). For administration of a pharmaceutical composition containing a compound of the disclosure, an amount of the pharmaceutical composition can be administered that contains an amount of the compound that is within one of the foregoing ranges. Likewise, for administration of one or more food products containing the compound, an amount of one or more food products can be administered that contain an amount of the compound that is within one of the foregoing ranges.

### EXAMPLES

### 6. Examples

Next, the present invention will be described more specifically with reference to Examples. However, Examples described below do not limit the scope of the present invention.

<Test Method>

(Elevated Plus-Maze (EPM) Test)

The elevated plus-maze consists of two open arms (25 cm×5 cm) and two closed arms (25 cm×5 cm×15 cm), and these arms are connected to a central platform elevated by 50 cm from the floor (see FIG. 1). In spite of such an elevated position, mice can walk safely in the closed arms because the closed arms are fenced. On the other hand, mice waking in the open arms feel anxious about falling from the elevated position because the open arms are open without being fenced. Therefore, a longer time when a mouse stays in the open arms means that mouse's feeling of anxiety is more alleviated, and serves as an index for an anxiolytic-like effect.

As shown in the right diagram of FIG. 1, a sample was administered to each mouse (ddY mouse, male, 23 to 27 g) 30 minutes before the test. 30 minutes later, the mouse was placed on the central platform facing one of the open arms to start the test. Cumulative time in the open arms was recorded during the 5-minute test time. The percentage of the time in the open arms was calculated as an index for an anxiolytic-like effect.

(Statistical Analysis)

The data obtained by the test was indicated by the sum of a mean and a standard error of the mean (SEM). The data shown in FIGS. 1 to 9, 17, 18, 20, and 21 was analyzed by one-way or two-way ANOVA. Subsequently, study was conducted for multiple comparison by the Tukey Kramer method. P-value of <0.05 is denoted in the drawings as “*”; p-value of <0.01 is denoted in the drawings as “**”.

### Production Example 1

(Extraction of RubisCo Protein)

Spinach was homogenized. The pH of the obtained homogenate was adjusted to pH 11 with a 1 N aqueous NaOH solution. The resulting homogenate was filtered through two pieces of gauze. The filtrate was centrifuged at 13,500 G at 5° C. for 50 minutes. After the centrifugation, the supernatant was filtered. The pH of the obtained filtrate was adjusted to pH 4.5 with acetic acid to precipitate the RubisCo protein. The precipitate was washed with acetone, ethanol and diethyl ether and dried in a pressure reducer to obtain a RubisCo powder.

The RubisCo protein thus extracted from spinach and a RubisCo protein preparation (Sigma-Aldrich Co. LLC) were analyzed by SDS-PAGE. The results are shown in FIG. 2.

(Enzymatic Digest)

Enzymatic digests of purified RubisCo protein were prepared under the following conditions.

The enzymes used, the mixing ratio between each enzyme and the protein and reaction conditions were as follows.

(i) Pepsin Digestion

Pepsin (Sigma-Aldrich Co. LLC): RubisCo=1:100 (weight ratio, final concentration of RubisCo: 0.99 mg/ml), reaction temperature: 37° C., reaction time: 5 hours; reaction buffer: pH 2.0.

(ii) Pepsin+Pancreatin Digestion

Enzymatic treatment was performed in the order of 1) and 2).

1) Pepsin (Sigma-Aldrich Co. LLC):RubisCo=1:100 (weight ratio, final concentration of Rubisco: 0.99 mg/ml), reaction temperature: 37° C., reaction time: 5 hours; reaction buffer: pH 2.0.

2) Pancreatin (Sigma-Aldrich Co. LLC):RubisCo=1:20 (weight ratio, final concentration of RubisCo: 0.86 mg/ml), reaction temperature: 37° C., reaction time: 5 hours; reaction buffer: pH 7.5.

After a lapse of the reaction time described above, the sample was boiled (100° ° C., 10 min) to terminate the enzymatic reaction.

### Production Example 2

(Peptide)

The peptides SYLPPLTT (SEQ ID NO: 1), YHIEPV (SEQ ID NO: 2), YLLVK (SEQ ID NO: 3) and SYLPPLT (SEQ ID NO: 4) were synthesized by standard methods, usually by a solid phase process based on an Fmoc-strategy.

### Experiments and Results

### Example 1: Elevated Plus-Maze Test (Enzymatic Digest)

A pepsin digest of RubisCo was orally administered as a sample at 3 mg/kg, 10 mg/kg or 30 mg/kg to each mouse, which was then used in the elevated plus-maze test (n=9 to 11). A vehicle physiological saline was administered alone and used as a control (the same holds true for the description below).

The results are shown in FIG. 3. The oral administration of the digest at all doses increased the ratio of an open arm residence time, with the result for the 10 mg/kg dose being statistically significant. This result indicates that the pepsin digest of RubisCo used as a sample has an anxiolytic-like effect when orally administered.

### Example 2: Elevated Plus-Maze Test (Enzymatic Digest)

A pepsin+pancreatin digest of RubisCo was orally administered as a sample at 3 mg/kg, 10 mg/kg or 30 mg/kg to each mouse, which was then used in the elevated plus-maze test (n=9 to 11).

The results are shown in FIG. 4. The oral administration of the digest at all doses increased the ratio of an open arm residence time, with the results for 3 mg/kg and 30 mg/kg doses being statistically significant. This result indicates that the pepsin+pancreatin digest of RubisCo used as a sample has an anxiolytic-like effect when orally administered.

### Example 3: Elevated Plus-Maze Test (Peptide)

The peptide SYLPPLTT (SEQ ID NO: 1) was orally administered as a sample at 0.3 mg/kg or 1 mg/kg to each mouse, which was then used in the elevated plus-maze test (n=13 to 16).

The results are shown in FIG. 5. The oral administration of the peptide SYLPPLTT (SEQ ID NO: 1) at both doses increased the ratio of an open arm residence time, with the result for the 1 mg/kg dose being statistically significant. This result indicates that the peptide SYLPPLTT (SEQ ID NO: 1) used as a sample has an anxiolytic-like effect when orally administered.

### Example 4: Elevated Plus-Maze Test (Peptide)

The peptide YHIEPV (SEQ ID NO: 2) was orally administered as a sample at 1 mg/kg, 3 mg/kg, or 10 mg/kg to each mouse, which was then used in the elevated plus-maze test (n=5 to 7).

The results are shown in FIG. 6. The oral administration of the peptide YHIEPV (SEQ ID NO: 2) at all doses increased the ratio of an open arm residence time, with the results for the 3 mg/kg and 10 mg/kg doses being statistically significant. This result indicates that the peptide YHIEPV (SEQ ID NO: 2) used as a sample has an anxiolytic-like effect when orally administered.

### Example 5: Elevated Plus-Maze Test (Peptide)

The peptide YLLVK (SEQ ID NO: 3) was orally administered as a sample at 0.03 mg/kg or 0.1 mg/kg to each mouse, which was then used in the elevated plus-maze test (n=5 to 7).

The results are shown in FIG. 7. The oral administration of the peptide YLLVK (SEQ ID NO: 3) at 0.03 mg/kg and 0.1 mg/kg significantly increased the ratio of an open arm residence time. This result indicates that the peptide YLLVK (SEQ ID NO: 3) used as a sample has an anxiolytic-like effect when orally administered.

### Example 6: Elevated Plus-Maze Test (Peptide)

The peptide SYLPPLT (SEQ ID NO: 4) was orally administered as a sample at 0.1 mg/kg, 0.3 mg/kg or 1 mg/kg to each mouse, which was then used in the elevated plus-maze test (n=5 to 13).

The results are shown in FIG. 8. The oral administration of the peptide SYLPPLT (SEQ ID NO: 4) at all doses increased the ratio of an open arm residence time, with the results for the 1 mg/kg dose being statistically significant. This result indicates that the peptide SYLPPLT (SEQ ID NO: 4) used as a sample has an anxiolytic-like effect when orally administered.

### Example 7: Study on Mechanism of Action Using Antagonist (Peptide)

The peptide SYLPPLTT (SEQ ID NO: 1) or the peptide SYLPPLT (SEQ ID NO: 4) (dose: 1.0 mg/kg) was used in combination with a serotonin 5-HT_{1A} receptor antagonist WAY100135 (dose: 10 mg/kg) and orally administered (p.o.) to each mouse, which was then used in the elevated plus-maze test (n=4 to 6).

The results are shown in FIG. 9. The combined use of either peptide with the inhibitor significantly decreased the ratio of an open arm residence time. This result indicates that the anxiolytic-like effect of the peptide is mediated by serotonin 5-HT_{1A} receptor.

In the drawings, the letter a or b represents that there is no significant difference (p<0.05) between means with the same letter.

### Reference Example 1

The proportions of the peptides SYLPPLTT (SEQ ID NO: 1), SYLPPLT (SEQ ID NO: 4), YHIEPV (SEQ ID NO: 2), and YLLVK (SEQ ID NO: 3) contained in the pepsin digest of RubisCo (in the table, Pepsin) and the pepsin+pancreatin digest of RubisCo (in the table, Pepsin→Pancreatin) were quantified by LC-MS.

LC-MS was conducted under the following conditions (manufactured by Waters Corp.).
LC: Acquity UPLC system
Column: Acquity BEH-C18
MS: Xevo Q-TOF.

The results are shown in Table 1. The numerical values are indicated by yield mol % and mass (ng)/1 mg digest.

TABLE 1
Peptide yield in enzymatic digest

| Peptide | Pepsin | Pepsin → Pancreatin |
|---|---|---|
| SYLPPLTT | 18.3 | ND |
| (SEQ ID NO: 1) | (2357) | |
| SYLPPLT | ND | 2.2 (253) |
| (SEQ ID NO: 4) | | |
| YHIEPV | ND | 15.0 (1643) |
| (SEQ ID NO: 2) | | |

Yield: mol % (ng/1 mg digest)

### Production Example 3

(Peptide)

The peptides FLLVK (SEQ ID NO: 5), WLLVK (SEQ ID NO: 6), YLL (SEQ ID NO:7), LVK (SEQ ID NO:9), YLLV (SEQ ID NO: 10), LLVK (SEQ ID NO: 11), YLVK (SEQ ID NO:12), YLLVR (SEQ ID NO:13), NYLLVKG (SEQ ID NO: 21), YLLAVK (SEQ ID NO:22), YLLNNK (SEQ ID NO:23), SYLPPL (SEQ ID NO:20) were synthesized by a standard method.

### Example 8: Elevated Plus-Maze Test (Peptide)

The peptide YLLVK(SEQ ID NO: 3), FLLVK (SEQ ID NO:5) and WLLVK (SEQ ID NO: 6) were orally administered as a sample at 0.1 mg/kg to each mouse, which was then used in the elevated plus-maze test (n=6).

The results are shown in FIG. 10. The oral administration of the peptide YLLVK (SEQ ID NO: 3), FLLVK (SEQ ID NO:5) and WLLVK (SEQ ID NO: 6) at 0.1 mg/kg increased the ratio of an open arm residence time. This result indicates that the peptide YLLVK (SEQ ID NO: 3), FLLVK (SEQ ID NO:5) and WLLVK (SEQ ID NO: 6) used as a sample has an anxiolytic-like effect when orally administered.

### Example 9: Elevated Plus-Maze Test (Peptide)

The peptide YLLVK (SEQ ID NO: 3), YLL (SEQ ID NO:7) and LVK (SEQ ID NO: 9) and LLVK (SEQ ID NO: 11) were orally administered as a sample at 0.1 mg/kg to each mouse, which was then used in the elevated plus-maze test (n=5).

The results are shown in FIG. 11. The oral administration of the peptide YLLVK (SEQ ID NO: 3), YLL (SEQ ID NO:7) and LVK (SEQ ID NO: 9) and LLVK (SEQ ID NO: 11) at 0.1 mg/kg increased the ratio of an open arm residence time. This result indicates that the peptide YLLVK (SEQ ID NO: 3), YLL (SEQ ID NO:7) and LVK (SEQ ID NO: 9) and LLVK (SEQ ID NO: 11) used as a sample has an anxiolytic-like effect when orally administered.

### Example 10: Elevated Plus-Maze Test (Peptide)

The peptide YLLVR (SEQ ID NO: 13), NYLLVKG (SEQ ID NO:21) and YLLAVK (SEQ ID NO: 22) and YLLNNK (SEQ ID NO: 23) were orally administered as a sample at 0.1 mg/kg to each mouse, which was then used in the elevated plus-maze test (n=5).

The results are shown in FIG. 12. The oral administration of the peptide YLL VR (SEQ ID NO: 13), NYLLVKG (SEQ ID NO:21) and YLLAVK (SEQ ID NO: 22) and YLLNNK (SEQ ID NO: 23) at 0.1 mg/kg increased the ratio of an open arm residence time. This result indicates that the peptide YLLVR (SEQ ID NO: 13), NYLLVKG (SEQ ID NO:21) and YLLAVK (SEQ ID NO: 22) and YLLNNK (SEQ ID NO: 23) used as a sample has an anxiolytic-like effect when orally administered.

### Example 11: Elevated Plus-Maze Test (Peptide)

The peptide YLLVK (SEQ ID NO: 3), YLLV (SEQ ID NO: 10) and YLVK (SEQ ID NO: 12) and YLLAVK (SEQ ID NO: 22) were orally administered as a sample at 0.1 mg/kg to each mouse, which was then used in the elevated plus-maze test (n=5).

The results are shown in FIG. 13. The oral administration of the peptide YLLVK (SEQ ID NO: 3), YLLV (SEQ ID NO:10) and YLVK (SEQ ID NO: 12) and YLLAVK (SEQ ID NO: 22) at 0.1 mg/kg increased the ratio of an open arm residence time. This result indicates that the peptide YLLVK (SEQ ID NO: 3), YLLV (SEQ ID NO:10) and YLVK (SEQ ID NO: 12) and YLLAVK (SEQ ID NO: 22) used as a sample has an anxiolytic-like effect when orally administered.

### Example 12: Elevated Plus-Maze Test (Peptide)

The peptide SYLPPL (SEQ ID NO:20) was orally administered as a sample at 0.3 mg/kg, 1.0 mg/kg or 10 mg/kg to each mouse, which was then used in the elevated plus-maze test (n=4 to 6).

The results are shown in FIG. 14. The oral administration of the peptide SYLPPL (SEQ ID NO:20) at 0.3 mg/kg, 1.0 mg/kg or 10 mg/kg increased the ratio of an open arm residence time. This result indicates that the peptide SYLPPL (SEQ ID NO:20) used as a sample has an anxiolytic-like effect when orally administered.

### Example 13: Elevated Plus-Maze Test (Peptide)

The peptide YLPPL (SEQ ID NO:14) was orally administered as a sample at 0.3 mg/kg, 3 mg/kg, and 10 mg/kg to each mouse, which was then used in the elevated plus-maze test (n=6).

The results are shown in FIG. 15. Without being bound by theory, it is believed that an N-terminal S on peptides comprising the sequence YLPPL (SEQ ID NO:14) increases activity compared to peptides lacking the N-terminal S.

### Example 14 Tail Suspension Test (Peptide)

The peptide SYLPPLT (SEQ ID NO: 4) was administered orally to mice (ddY mice, males, 24˜30 g) at 0.03 mg/kg, 0.1 mg/kg or 0.3 mg/kg and 30 minutes later the mice were suspended by their tails for six minutes.(n=11 to 14)

The results are shown in FIG. 16. A decrease in immobility time was observed for the mice administered the peptide SYLPPLT (SEQ ID NO:4) at 0.03 mg/kg, 0.1 mg/kg or 0.3 mg/kg.

This result indicates that the peptide SYLPPLT (SEQ ID NO: 4) used as a sample has an antidepressant-like effect when orally administered.

### Example 15: Elevated Plus-Maze Test (Enzymatic Digest)

A pepsin digest of RubisCo was orally administered as a sample at 10 mg/kg or 30 mg/kg to each mouse, which was then used in the elevated plus-maze test (n=18 to 19). A pepsin-pancreatin digest of RubisCo was orally administered as a sample at 3 mg/kg, 10 mg/kg or 30 mg/kg to each mouse, which was then used in the elevated plus-maze test (n=6-14).

The results are shown in FIG. 17. The oral administration of the digests at all doses increased the ratio of an open arm residence time. This result indicates that the pepsin and pepsin+pancreatin digests of RubisCo used as samples have an anxiolytic-like effect when orally administered.

### Example 16: Elevated Plus-Maze Test (Peptide)

The peptides SYLPPLTT (SEQ ID NO: 1), SYLPPLT (SEQ ID NO:4) and YHIEPV (SEQ ID NO:2) were orally administered as a sample at (i) 0.3 mg/kg and 1 mg/kg, (ii) 0.1 mg/kg, 0.3 mg/kg, and 1 mg/kg, and (iii) 1 mg/kg, 3 mg/kg, and 10 mg/kg, respectively to mice, which were then used in the elevated plus-maze test (n=13-16, 5-13, and 5-6, respectively). Diazepam was orally administered as a sample at 1 mg/kg, 3 mg/kg, and 10 mg/kg to mice, which were then used in the elevated plus-maze test (n=11-12), and was tested as a comparator compound.

The results are shown in FIG. 18. The oral administration of the peptides SYLPPLTT (SEQ ID NO: 1), SYLPPLT (SEQ ID NO:4) and YHIEPV (SEQ ID NO:2) and YLLAVK (SEQ ID NO: 22) increased the ratio of an open arm residence time. At some doses, the increase in open arm residence time was greater than the increase observed for diazepam. This result indicates that the peptide SYLPPLTT (SEQ ID NO: 1), SYLPPLT (SEQ ID NO:4) and YHIEPV (SEQ ID NO:2) and YLLAVK (SEQ ID NO: 22) used as a sample has an anxiolytic-like effect when orally administered.

### Example 17: Spinach RubisCo Cleavage Sites

Cleavage sites in spinach RubisCo small subunit and large subunit by gastrointestinal proteases leading to release rALPs were determined. The cleavage sites in the small subunit (A) and large subunit (B) are shown in FIG. 19. Solid arrows indicate cleavage sites.

### Example 18: Elevated Plus-Maze Test (Peptide)

The peptide YHIEPV (SEQ ID NO:2) was orally administered as a sample to mice 30 minutes before an elevated plus-maze test, with or without WAY100135, an antagonist selective for 5-HT_{1A} receptor (n=13-15). WAY100135 was administered 50 minutes before the elevated plus-maze test. Separately, the peptide YHIEPV (SEQ ID NO:2) was orally administered as a sample to mice 30 minutes before an elevated plus-maze test, with or without naltrindole, an 8-opioid receptor antagonist, which was administered 50 minutes before the elevated plus-maze test (n=11-15).

The results are shown in FIG. 20. The results indicates that the anxiolytic-like effect of peptide SYLPPLTT (SEQ ID NO: 1) and SYLPPLT (SEQ ID NO:4) are mediated by activation of serotonin 5-HT_{1A} receptor, while the anxiolytic-like effect of peptide YHIEPV (SEQ ID NO:2) is not mediated by activation of serotonin 5-HTopio_{1A} receptor, but is mediated by activation 8-opioid receptor.

### (Example 19: Intracellular CAMP Elevation (Peptide))

It is reported that 5-HT_{1A} receptor and δ opioid receptor act via G protein to inhibit adenylyl cyclase. The peptides SYLPPLTT (SEQ ID NO:1), SYLPPLT (SEQ ID NO:4) and YHIEPV (SEQ ID NO:2) were applied to Neuro-2a cells at concentrations of 0.3 mM and 1 mM in the presence of forskolin (FSK) to assess whether the peptides would suppress forskolin-stimulated intracellular cAMP elevation

The results are shown in FIG. 21. The results suggest that the peptides suppress forskolin-stimulated intracellular cAMP elevation.

### 7. Specific Embodiments

The present disclosure is exemplified by the specific embodiments below.
1. A compound that is a peptide or a salt thereof, wherein the peptide:
(a) is 3 to 20 amino acids in length;
(b) has an amino acid sequence comprising at least 3 consecutive amino acids from the N-terminal or C-terminal end of the amino acid sequence X₁LX₂X₃VK (SEQ ID NO:8), wherein
(i) X₁ is a hydrophobic amino acid,
(ii) X₂ and X₃ are each independently selected from any amino acid;
(c) has a hydrophobic N-terminal amino acid; and
(d) does not consist of the amino acid sequence YLLVR (SEQ ID NO:13), X₄LX₅ (SEQ ID NO:30), X₄LX₅EIAR (SEQ ID NO:31), VYLPR (SEQ ID NO:32), YLPR (SEQ ID NO:33), or VLQRF (SEQ ID NO:34), where X₄ is Y, F, W, or H and X₅ is Y, F, W, Q, or L.
2. The compound of embodiment 1, wherein X₁ is an aromatic amino acid.
3. The compound of embodiment 2, wherein X₁ is Y.
4. The compound of embodiment 2, wherein X₁ is F.
5. The compound of embodiment 2, wherein X₁ is W.
6. The compound of embodiment 1, wherein X₁ is A.
7. The compound of embodiment 1, wherein X₁ is I.
8. The compound of embodiment 1, wherein X₁ is L.
9. The compound of embodiment 1, wherein X₁ is V.
10. The compound of embodiment 1, wherein X₁ is M.
11. The compound of any one of embodiments 1 to 10, wherein X₂ is a hydrophobic amino acid.
12. The compound of embodiment 11, wherein X₂ is selected from L, I, V, and A.
13. The compound of embodiment 12, wherein X₂ is L.
14. The compound of embodiment 12, wherein X₂ is I.
15. The compound of embodiment 12, wherein X₂ is V.
16. The compound of embodiment 12, wherein X₂ is A.
17. The compound of any one of embodiments 1 to 16, wherein X₃ is a hydrophobic amino acid.
18. The compound of embodiment 17, wherein X₃ is selected from L, I, V, and A.
19. The compound of embodiment 18, wherein X₃ is L.
20. The compound of embodiment 18, wherein X₃ is I.
21. The compound of embodiment 18, wherein X₃ is V.
22. The compound of embodiment 18, wherein X₃ is A.
23. The compound of any one of embodiments 1 to 22, wherein the peptide comprises the amino acid sequence X₁LX₂ (SEQ ID NO:15).
24. The compound of embodiment 23, wherein X₁ is the N-terminal amino acid of the peptide.
25. The compound of embodiment any one of embodiments 1 to 22, wherein the peptide comprises the amino acid sequence X₃VK (SEQ ID NO:16).
26. The compound of embodiment 1, wherein the peptide comprises or consists of the amino acid sequence YLL (SEQ ID NO:7).
27. The compound of embodiment 1, wherein the peptide comprises or consists of the amino acid sequence LVK (SEQ ID NO:9).
28. The compound of any one of embodiments 1 to 22, wherein the peptide comprises the amino acid sequence X₁LX₂X: (SEQ ID NO:17).
29. The compound of embodiment 28, wherein the peptide comprises the amino acid sequence X₁LX₂X₃K (SEQ ID NO:29).
30. The compound of embodiment 28 or embodiment 29, wherein X₁ is the N-terminal amino acid of the peptide.
31. The compound of any one of embodiments 1 to 22, wherein the peptide comprises the amino acid sequence X₂X₃VK (SEQ ID NO:18).
32. The compound of embodiment 1, wherein the peptide comprises or consists of the amino acid sequence YLLV (SEQ ID NO:10).
33. The compound of embodiment 1, wherein the peptide comprises or consists of the amino acid sequence LLVK (SEQ ID NO:11).
34. The compound of embodiment 1, wherein the peptide comprises or consists of the amino acid sequence YLVK (SEQ ID NO:12).
35. The compound of any one of embodiments 1 to 22, wherein the peptide comprises the amino acid sequence X₁LX₂X₃V (SEQ ID NO:19).
36. The compound of embodiment 35, wherein X₁ is the N-terminal amino acid of the peptide.
37. The compound of any one of embodiments 1 to 22, wherein the peptide comprises the amino acid sequence LX₂X₃VK (SEQ ID NO:27).
38. The compound of embodiment 1, wherein the peptide comprises or consists of the amino acid sequence YLLVK (SEQ ID NO:3).
39. The compound of embodiment 1, wherein the peptide comprises or consists of the amino acid sequence FLLVK (SEQ ID NO:5).
40. The compound of embodiment 1, wherein the peptide comprises or consists of the amino acid sequence WLLVK (SEQ ID NO:6).
41. The compound of embodiment 1, wherein the peptide comprises the amino acid sequence YLLVR (SEQ ID NO:13).
42. The compound of any one of embodiments 1 to 22, wherein the peptide comprises the amino acid sequence X₁LX₂X₃VK (SEQ ID NO:8).
43. The compound of embodiment 1, wherein the peptide comprises or consists of the amino acid sequence YLLAVK (SEQ ID NO:22).
44. The compound of embodiment 1, wherein the peptide comprises or consists of the amino acid sequence YLLNNK (SEQ ID NO:22).
45. The compound of any one of embodiments 1 to 44, wherein the peptide is 3 to 15 amino acids in length.
46. The compound of any one of embodiments 1 to 44, wherein the peptide is 3 to 10 amino acids in length.
47. The compound of any one of embodiments 1 to 44, wherein the peptide is 4 to 20 amino acids in length.
48. The compound of any one of embodiments 1 to 44, wherein the peptide is 4 to 15 amino acids in length.
49. The compound of any one of embodiments 1 to 44, wherein the peptide is 4 to 10 amino acids in length.
50. The compound of any one of embodiments 1 to 44, wherein the peptide is 5 to 20 amino acids in length.
51. The compound of any one of embodiments 1 to 44, wherein the peptide is 5 to 15 amino acids in length.
52. The compound of any one of embodiments 1 to 44, wherein the peptide is 5 to 10 amino acids in length.
53. The compound of any one of embodiments 1 to 27, wherein the peptide is 3 amino acids in length.
54. The compound of any one of embodiments 1 to 34, wherein the peptide is 4 amino acids in length.
55. The compound of any one of embodiments 1 to 40, wherein the peptide is 5 amino acids in length.
56. The compound of any one of embodiments 1 to 44, wherein the peptide is 6 amino acids in length.
57. The compound of any one of embodiments 1 to 44, wherein the peptide is 7 amino acids in length.
58. The compound of any one of embodiments 1 to 44, wherein the peptide is 8 amino acids in length.
59. The compound of any one of embodiments 1 to 44, wherein the peptide is 9 amino acids in length.
60. The compound of any one of embodiments 1 to 44, wherein the peptide is 10 amino acids in length.
61. The compound of any one of embodiments 1 to 44, wherein the peptide is 11 amino acids in length.
62. The compound of any one of embodiments 1 to 44, wherein the peptide is 12 amino acids in length.
63. The compound of any one of embodiments 1 to 44, wherein the peptide is 13 amino acids in length.
64. The compound of any one of embodiments 1 to 44, wherein the peptide is 14 amino acids in length.
65. The compound of any one of embodiments 1 to 44, wherein the peptide is 15 amino acids in length.
66. The compound of any one of embodiments 1 to 44, wherein the peptide is 16 amino acids in length.
67. The compound of any one of embodiments 1 to 44, wherein the peptide is 17 amino acids in length.
68. The compound of any one of embodiments 1 to 44, wherein the peptide is 18 amino acids in length.
69. The compound of any one of embodiments 1 to 44, wherein the peptide is 19 amino acids in length.
70. The compound of any one of embodiments 1 to 44, wherein the peptide is 20 amino acids in length.
71. A compound that is a peptide or a salt thereof, wherein the peptide:
(a) is 5 to 20 amino acids in length; and
(b) comprises the amino acid sequence SYLPP (SEQ ID NO:24) at the N-terminal end of the peptide.
72. The compound of embodiment 71, wherein the peptide comprises or consists of the amino acid sequence SYLPPL (SEQ ID NO:20).
73. The compound of embodiment 71, wherein the peptide comprises or consists of the amino acid sequence SYLPPLT (SEQ ID NO:4).
74. The compound of embodiment 71, wherein the peptide comprises or consists of the amino acid sequence SYLPPLTT (SEQ ID NO:1).
75. The peptide of any one of embodiments 71 to 74, wherein the peptide is 5 to 15 amino acids in length.
76. The peptide of any one of embodiments 71 to 74, wherein the peptide is 5 to 10 amino acids in length.
77. The compound of embodiment 71, wherein the peptide is 5 amino acids in length.
78. The compound of embodiment 71 or embodiment 72, wherein the peptide is 6 amino acids in length.
79. The compound of any one of embodiments 71 to 73, wherein the peptide is 7 amino acids in length.
80. The compound of any one of embodiments 71 to 74, wherein the peptide is 8 amino acids in length.
81. The compound of any one of embodiments 71 to 74, wherein the peptide is 9 amino acids in length.
82. The compound of any one of embodiments 71 to 74, wherein the peptide is 10 amino acids in length.
83. The compound of any one of embodiments 71 to 74, wherein the peptide is 11 amino acids in length.
84. The compound of any one of embodiments 71 to 74, wherein the peptide is 12 amino acids in length.
85. The compound of any one of embodiments 71 to 74, wherein the peptide is 13 amino acids in length.
86. The compound of any one of embodiments 71 to 74, wherein the peptide is 14 amino acids in length.
87. The compound of any one of embodiments 71 to 74, wherein the peptide is 15 amino acids in length.
88. The compound of any one of embodiments 71 to 74, wherein the peptide is 16 amino acids in length.
89. The compound of any one of embodiments 71 to 74, wherein the peptide is 17 amino acids in length.
90. The compound of any one of embodiments 71 to 74, wherein the peptide is 18 amino acids in length.
91. The compound of any one of embodiments 71 to 74, wherein the peptide is 19 amino acids in length.
92. The compound of any one of embodiments 71 to 74, wherein the peptide is 20 amino acids in length.
93. A compound that is a peptide or a salt thereof, wherein the peptide:
(a) is 6 to 20 amino acids in length; and
(b) comprises the amino acid sequence YHIEPV (SEQ ID NO:2).
94. The compound of embodiment 93, wherein the peptide comprises the amino acid sequence YHIEPV (SEQ ID NO:2) at the N-terminal end of the peptide.
95. The compound of embodiment 93, wherein the peptide comprises the amino acid sequence YHIEPV (SEQ ID NO:2) at the C-terminal end of the peptide.
96. The compound of embodiment 93, wherein the peptide consists of the amino acid sequence YHIEPV (SEQ ID NO:2).
97. The compound of any one of embodiments 93 to 96, wherein the peptide is 6 to 15 amino acids in length.
98. The compound of any one of embodiments 93 to 96, wherein the peptide is 6 to 10 amino acids in length.
99. The compound of any one of embodiments 93 to 96, wherein the peptide is 7 amino acids in length.
100. The compound of any one of embodiments 93 to 96, wherein the peptide is 8 amino acids in length.
101. The compound of any one of embodiments 93 to 96, wherein the peptide is 9 amino acids in length.
102. The compound of any one of embodiments 93 to 96, wherein the peptide is 10 amino acids in length.
103. The compound of any one of embodiments 93 to 96, wherein the peptide is 11 amino acids in length.
104. The compound of any one of embodiments 93 to 96, wherein the peptide is 12 amino acids in length.
105. The compound of any one of embodiments 93 to 96, wherein the peptide is 13 amino acids in length.
106. The compound of any one of embodiments 93 to 96, wherein the peptide is 14 amino acids in length.
107. The compound of any one of embodiments 93 to 96, wherein the peptide is 15 amino acids in length.
108. The compound of any one of embodiments 93 to 96, wherein the peptide is 16 amino acids in length.
109. The compound of any one of embodiments 93 to 96, wherein the peptide is 17 amino acids in length.
110. The compound of any one of embodiments 93 to 96, wherein the peptide is 18 amino acids in length.
111. The compound of any one of embodiments 93 to 96, wherein the peptide is 19 amino acids in length.
112. The compound of any one of embodiments 93 to 96, wherein the peptide is 20 amino acids in length.
113. A compound that is a conjugate or a salt thereof, the conjugate comprising:
(a) a peptide moiety that:
(i) is 3 to 20 amino acids in length;
(ii) has an amino acid sequence comprising at least 3 consecutive amino acids from the N-terminal or C-terminal end of the amino acid sequence X₁LX₂X₃VK (SEQ ID NO:8), wherein
X₁ is a hydrophobic amino acid,
X₂ and X₃ are each independently selected from any amino acid;
attached to
(b) one or more conjugate moieties.
114. The compound of embodiment 113, wherein the peptide moiety does not consist of the amino acid sequence YLLVR (SEQ ID NO:13), X₄LX₅ (SEQ ID NO:30), X₄LX₅EIAR (SEQ ID NO:31), VYLPR (SEQ ID NO:32), YLPR (SEQ ID NO:33), or VLQRF (SEQ ID NO:34), where X₄ is Y, F, W, or H and X₅ is Y, F, W, Q, or L.
115. The compound of embodiment 113 or embodiment 114, wherein X₁ is an aromatic amino acid.
116. The compound of embodiment 115, wherein X₁ is Y.
117. The compound of embodiment 115, wherein X₁ is F.
118. The compound of embodiment 115, wherein X₁ is W.
119. The compound of embodiment 113, wherein X₁ is A.
120. The compound of embodiment 113, wherein X₁ is I.
121. The compound of embodiment 113, wherein X₁ is L.
122. The compound of embodiment 113, wherein X₁ is V.
123. The compound of embodiment 113, wherein X₁ is M.
124. The compound of any one of embodiments 113 to 123, wherein X₂ is a hydrophobic amino acid.
125. The compound of embodiment 124, wherein X₂ is selected from L, I, V, and A.
126. The compound of embodiment 125, wherein X₂ is L.
127. The compound of embodiment 125, wherein X₂ is I.
128. The compound of embodiment 125, wherein X₂ is V.
129. The compound of embodiment 125, wherein X₂ is A.
130. The compound of any one of embodiments 113 to 129, wherein X₃ is a hydrophobic amino acid.
131. The compound of embodiment 130, wherein X₃ is selected from L, I, V, and A.
132. The compound of embodiment 131, wherein X₃ is L.
133. The compound of embodiment 131, wherein X₃ is I.
134. The compound of embodiment 131, wherein X₃ is V.
135. The compound of embodiment 131, wherein X₃ is A.
136. The compound of any one of embodiments 113 to 135, wherein the peptide moiety comprises the amino acid sequence X₁LX₂ (SEQ ID NO:15).
137. The compound of embodiment 136, wherein X₁ is the N-terminal amino acid of the peptide moiety.
138. The compound of embodiment any one of embodiments 113 to 135, wherein the peptide moiety comprises the amino acid sequence X₃VK (SEQ ID NO:16).
139. The compound of embodiment 113, wherein the peptide moiety comprises or consists of the amino acid sequence YLL (SEQ ID NO:7).
140. The compound of embodiment 113, wherein the peptide moiety comprises or consists of the amino acid sequence LVK (SEQ ID NO:9).
141. The compound of any one of embodiments 113 to 135, wherein the peptide moiety comprises the amino acid sequence X₁LX₂X₃ (SEQ ID NO:17).
142. The compound of embodiment 141, wherein the peptide moiety comprises the amino acid sequence X₁LX₂X₃K (SEQ ID NO:29).
143. The compound of embodiment 141 or embodiment 142, wherein X₁ is the N-terminal amino acid of the peptide.
144. The compound of any one of embodiments 113 to 135, wherein the peptide moiety comprises the amino acid sequence X₂X₃VK (SEQ ID NO:18).
145. The compound of embodiment 113, wherein the peptide moiety comprises or consists of the amino acid sequence YLLV (SEQ ID NO:10).
146. The compound of embodiment 113, wherein the peptide moiety comprises or consists of the amino acid sequence LL VK (SEQ ID NO:11).
147. The compound of embodiment 113, wherein the peptide moiety comprises or consists of the amino acid sequence YLVK (SEQ ID NO:12).
148. The compound of any one of embodiments 113 to 135, wherein the peptide moiety comprises the amino acid sequence X₁LX₂X₃V (SEQ ID NO:19).
149. The compound of embodiment 148, wherein X₁ is the N-terminal amino acid of the peptide moiety.
150. The compound of any one of embodiments 113 to 135, wherein the peptide moiety comprises the amino acid sequence LX₂X₃VK (SEQ ID NO:27).
151. The compound of embodiment 113, wherein the peptide moiety comprises or consists of the amino acid sequence YLLVK (SEQ ID NO:3).
152. The compound of embodiment 113, wherein the peptide moiety comprises or consists of the amino acid sequence FLLVK (SEQ ID NO:5).
153. The compound of embodiment 113, wherein the peptide moiety comprises or consists of the amino acid sequence WLLVK (SEQ ID NO:6).
154. The compound of embodiment 113, wherein the peptide moiety comprises the amino acid sequence YLLVR (SEQ ID NO:13).
155. The compound of any one of embodiments 113 to 135, wherein the peptide moiety comprises the amino acid sequence X₁LX₂X₃VK (SEQ ID NO:8).
156. The compound of embodiment 113, wherein the peptide moiety comprises or consists of the amino acid sequence YLLAVK (SEQ ID NO:22).
157. The compound of embodiment 113, wherein the peptide moiety comprises or consists of the amino acid sequence YLLNNK (SEQ ID NO:22).
158. The compound of any one of embodiments 113 to 157, wherein the peptide moiety is 3 to 15 amino acids in length.
159. The compound of any one of embodiments 113 to 157, wherein the peptide moiety is 3 to 10 amino acids in length.
160. The compound of any one of embodiments 113 to 157, wherein the peptide moiety is 4 to 20 amino acids in length.
161. The compound of any one of embodiments 113 to 157, wherein the peptide moiety is 4 to 15 amino acids in length.
162. The compound of any one of embodiments 113 to 157, wherein the peptide moiety is 4 to 10 amino acids in length.
163. The compound of any one of embodiments 113 to 157, wherein the peptide moiety is 5 to 20 amino acids in length.
164. The compound of any one of embodiments 113 to 157, wherein the peptide moiety is 5 to 15 amino acids in length.
165. The compound of any one of embodiments 113 to 157, wherein the peptide moiety is 5 to 10 amino acids in length.
166. The compound of any one of embodiments 113 to 140, wherein the peptide moiety is 3 amino acids in length.
167. The compound of any one of embodiments 113 to 147, wherein the peptide moiety is 4 amino acids in length.
168. The compound of any one of embodiments 113 to 154, wherein the peptide moiety is 5 amino acids in length.
169. The compound of any one of embodiments 113 to 157, wherein the peptide moiety is 6 amino acids in length.
170. The compound of any one of embodiments 113 to 157, wherein the peptide moiety is 7 amino acids in length.
171. The compound of any one of embodiments 113 to 157, wherein the peptide moiety is 8 amino acids in length.
172. The compound of any one of embodiments 113 to 157, wherein the peptide moiety is 9 amino acids in length.
173. The compound of any one of embodiments 113 to 157, wherein the peptide moiety is 10 amino acids in length.
174. The compound of any one of embodiments 113 to 157, wherein the peptide moiety is 11 amino acids in length.
175. The compound of any one of embodiments 113 to 157, wherein the peptide moiety is 12 amino acids in length.
176. The compound of any one of embodiments 113 to 157, wherein the peptide moiety is 13 amino acids in length.
177. The compound of any one of embodiments 113 to 157, wherein the peptide moiety is 140amino acids in length.
178. The compound of any one of embodiments 113 to 157, wherein the peptide moiety is 15 amino acids in length.
179. The compound of any one of embodiments 113 to 157, wherein the peptide moiety is 16 amino acids in length.
180. The compound of any one of embodiments 113 to 157, wherein the peptide moiety is 17 amino acids in length.
181. The compound of any one of embodiments 113 to 157, wherein the peptide moiety is 18 amino acids in length.
182. The compound of any one of embodiments 113 to 157, wherein the peptide moiety is 19 amino acids in length.
183. The compound of any one of embodiments 113 to 157, wherein the peptide moiety is 20 amino acids in length.
184. The compound of any one of embodiments 113 to 183, wherein the peptide moiety has a hydrophobic N-terminal amino acid.
185. A compound that is a conjugate or a salt thereof, the conjugate comprising a peptide moiety consisting of the peptide of any one of embodiments 71 to 112 and one or more conjugate moieties.
186. The compound of any one of embodiments 113 to 185, wherein at least one of the one or more conjugate moieties comprises a polymer, an amino group, an acyl group, an alkyl group, a phosphate group, a lipid or a sugar.
187. The compound of embodiment 186, wherein at least one of the one or more conjugate moieties comprises a polymer.
188. The compound of embodiment 187, wherein the polymer comprises a polyethylene glycol, polyvinyl pyrrolidone, polylactic-co-glycolic acid, N-(2-hydroxypropyl) methacrylamide copolymer, polyglutamic acid, or a polysaccharide.
189. The compound of any one of embodiments 186 to 188, wherein at least one of the one or more conjugate moieties comprises an amine group.
190. The compound of embodiment 189, wherein the amine group is an amino group, an alkyl amino group, or a dialkyl amino group.
191. The compound of any one of embodiments 186 to 190, wherein at least one of the one or more conjugate moieties comprises an acyl group.
192. The compound of embodiment 191, wherein the acyl group is a formyl group or an acetyl group.
193. The compound of any one of embodiments 186 to 192, wherein at least one of the one or more conjugate moieties comprises an alkyl group.
194. The compound of embodiment 193, wherein the alkyl group is a methyl group or an ethyl group.
195. The compound of any one of embodiments 186 to 194, wherein at least one of the one or more conjugate moieties comprises a phosphate group.
196. The compound of any one of embodiments 186 to 195, wherein at least one of the one or more conjugate moieties comprises a lipid.
197. The compound of any one of embodiments 186 to 196, wherein at least one of the one or more conjugate moieties comprises a sugar.
198. The compound of any one of embodiments 113 to 190 or, to the extent dependent from embodiment 186, the compound of any one of embodiments 191 to 197, which comprises a single conjugate moiety.
199. The compound of embodiment 198, wherein the conjugate moiety is attached to the N-terminal amino acid of the peptide moiety.
200. The compound of embodiment 198, wherein the conjugate moiety is attached to the C-terminal amino acid of the peptide moiety.
201. The compound of any one of embodiments 113 to 197, which comprises more than one conjugate moiety.
202. The compound of embodiment 201, wherein all of the conjugate moieties are the same.
203. The compound of embodiment 201, wherein not all of the conjugate moieties are the same.
204. The compound of embodiment 201, wherein all of the conjugate moieties are different.
205. The compound of embodiment 201, which comprises a conjugate moiety attached to the N-terminal amino acid of the peptide moiety and a conjugate moiety attached to the C-terminal amino acid of the peptide moiety.
206. The compound of embodiment 205, wherein the conjugate moiety attached to the N-terminal amino acid of the peptide moiety is the same as the conjugate moiety attached to the C-terminal amino acid of the peptide moiety.
207. The compound of embodiment 205, wherein the conjugate moiety attached to the N-terminal amino acid of the peptide moiety is different from the conjugate moiety attached to the C-terminal amino acid of the peptide moiety.
208. A compound that is a peptide or a salt thereof, wherein the peptide consists of the amino acid sequence YLLVR (SEQ ID NO:13), for treating a mood disorder, an anxiety disorder, or a disorder of diminished motivation.
209. The compound of any one of embodiments 1 to 208, which is a salt.
210. The compound of embodiment 209, wherein the salt is an acid addition salt.
211. The compound of embodiment 210, wherein the acid is:
(a) hydrochloric acid;
(b) sulfuric acid;
(c) nitric acid;
(d) phosphoric acid;
(e) hydrobromic acid;
(f) perchloric acid;
(g) citric acid;
(h) succinic acid;
(i) maleic acid;
(j) fumaric acid;
(k) malic acid;
(l) tartaric acid;
(m) p-toluenesulfonic acid;
(n) benzenesulfonic acid;
(o) methanesulfonic acid; or
(p) trifluoroacetic acid.
212. The compound of embodiment 209, wherein the salt is a base addition salt.
213. The compound of embodiment 212, wherein the base is:
(a) sodium hydroxide;
(b) potassium hydroxide;
(c) lithium hydroxide;
(d) calcium hydroxide; or
(e) magnesium hydroxide.
214. A pharmaceutical composition comprising the compound of any one of embodiments 1 to
213 and one or more pharmaceutically acceptable carriers, diluents and/or excipients, optionally which is formulated for oral administration.
215. A food product comprising as an additive the compound of any one of embodiments 1 to 213.
216. The food product of embodiment 215, which is a dietary supplement.
217. The food product of embodiment 215, which is a functional food.
218. A method for treating a subject suffering from a mood disorder, an anxiety disorder, or a disorder of diminished motivation, comprising administering to the subject an effective amount of the compound of any one of embodiments 1 to 213 or the pharmaceutical composition of embodiment 214.
219. The method of embodiment 218, which comprises treating a subject suffering from a mood disorder.
220. The method of embodiment 219, wherein the mood disorder comprises depression, bipolar disorder, or adjustment disorder.
221. The method of embodiment 220, which comprises treating a subject suffering from depression.
222. The method of embodiment 220, which comprises treating a subject suffering from bipolar disorder
223. The method of embodiment 220, which comprises treating a subject suffering from adjustment disorder.
224. The method of embodiment 218, which comprises treating a subject suffering from an anxiety disorder.
225. The method of embodiment 218, which comprises treating a subject suffering from a disorder of diminished motivation.
226. The method of embodiment 225, wherein the disorder of diminished motivation comprises apathy, abulia, or akinetic mutism.
227. The method of embodiment 226, which comprises treating a subject suffering from apathy.
228. The method of embodiment 226, which comprises treating a subject suffering from abulia.
229. The method of embodiment 226, which comprises treating a subject suffering from akinetic mutism.
230. The method of any one of embodiments 218 to 229, wherein the compound or pharmaceutical composition is administered orally.
231. The method of embodiment 230, wherein the amount of the compound administered to the subject per day is, or the amount of the pharmaceutical composition administered to the subject per day contains, 0.005 mg/kg to 500 mg/kg of the compound.
232. The method of embodiment 230, wherein the amount of the compound administered to the subject per day is, or the amount of the pharmaceutical composition administered to the subject per day contains, 0.005 mg/kg to 100 mg/kg of the compound.
233. The method of embodiment 230, wherein the amount of the compound administered to the subject per day is, or the amount of the pharmaceutical composition administered to the subject per day contains, 0.005 mg/kg to 30 mg/kg of the compound.
234. The method of embodiment 230, wherein the amount of the compound administered to the subject per day is, or the amount of the pharmaceutical composition administered to the subject per day contains, 0.005 mg/kg to 1 mg/kg of the compound.
235. The method of embodiment 230, wherein the amount of the compound administered to the subject per day is, or the amount of the pharmaceutical composition administered to the subject per day contains, 0.01 mg/kg to 30 mg/kg of the compound.
236. The method of embodiment 230, wherein the amount of the compound administered to the subject per day is, or the amount of the pharmaceutical composition administered to the subject per day contains, 0.01 mg/kg to 3 mg/kg of the compound.
237. The method of embodiment 230, wherein the amount of the compound administered to the subject per day is, or the amount of the pharmaceutical composition administered to the subject per day contains, 0.01 mg/kg to 1 mg/kg of the compound.
238. The method of embodiment 230, wherein the amount of the compound administered to the subject per day is, or the amount of the pharmaceutical composition administered to the subject per day contains, 0.02 mg/kg to 5 mg/kg of the compound.
239. The method of embodiment 230, wherein the amount of the compound administered to the subject per day is, or the amount of the pharmaceutical composition administered to the subject per day contains, 0.02 mg/kg to 2 mg/kg of the compound.
240. The method of embodiment 230, wherein the amount of the compound administered to the subject per day is, or the amount of the pharmaceutical composition administered to the subject per day contains, 0.02 mg/kg to 1 mg/kg of the compound.
241. The method of embodiment 230, wherein the amount of the compound administered to the subject per day is, or the amount of the pharmaceutical composition administered to the subject per day contains, 0.1 mg to 50 g.
242. The method of embodiment 230, wherein the amount of the compound administered to the subject per day is, or the amount of the pharmaceutical composition administered to the subject per day contains, 0.1 mg to 10 g of the compound.
243. The method of embodiment 230, wherein the amount of the compound administered to the subject per day is, or the amount of the pharmaceutical composition administered to the subject per day contains, 0.1 mg to 3 g of the compound.
244. The method of embodiment 230, wherein the amount of the compound administered to the subject per day is, or the amount of the pharmaceutical composition administered to the subject per day contains, 0.1 mg to 100 mg of the compound.
245. The method of embodiment 230, wherein the amount of the compound administered to the subject per day is, or the amount of the pharmaceutical composition administered to the subject per day contains, 0.1 mg to 1 mg of the compound.
246. The method of embodiment 230, wherein the amount of the compound administered to the subject per day is, or the amount of the pharmaceutical composition administered to the subject per day contains, 0.3 mg to 3 g of the compound.
247. The method of embodiment 230, wherein the amount of the compound administered to the subject per day is, or the amount of the pharmaceutical composition administered to the subject per day contains, 0.3 mg to 100 mg of the compound.
248. A method for treating or preventing a mood disorder, an anxiety disorder, or a disorder of diminished motivation, comprising administering to a subject prone to or suffering from a mood disorder, an anxiety disorder, or a disorder of diminished motivation an effective amount of one or more food products of any one of embodiments 215 to 217.
249. The method of embodiment 248, which comprises treating or preventing a mood disorder.
250. The method of embodiment 249, wherein the mood disorder comprises depression, bipolar disorder, or adjustment disorder.
251. The method of embodiment 250, which comprises treating or preventing depression.
252. The method of embodiment 250, which comprises treating or preventing bipolar disorder.
253. The method of embodiment 250, which comprises treating or preventing adjustment disorder.
254. The method of embodiment 248, which comprises treating or preventing an anxiety disorder.
255. The method of embodiment 248, which comprises treating or preventing a disorder of diminished motivation.
256. The method of embodiment 255, wherein the disorder of diminished motivation comprises apathy, abulia, or akinetic mutism.
257. The method of embodiment 256, which comprises treating or preventing apathy.
258. The method of embodiment 256, which comprises treating or preventing abulia.
259. The method of embodiment 256, which comprises treating or preventing akinetic mutism.
260. The method of any one of embodiments 248 to 259, wherein the amount of the one or more food products administered to the subject per day contains 0.005 mg/kg to 500 mg/kg of the compound.
261. The method of embodiment 260, wherein the amount of the one or more food products administered to the subject per day contains 0.005 mg/kg to 100 mg/kg of the compound.
262. The method of embodiment 260, wherein the amount of the one or more food products administered to the subject per day contains 0.005 mg/kg to 30 mg/kg of the compound.
263. The method of embodiment 260, wherein the amount of the one or more food products administered to the subject per day contains 0.005 mg/kg to 1 mg/kg of the compound.
264. The method of embodiment 260, wherein the amount of the one or more food products administered to the subject per day contains 0.01 mg/kg to 30 mg/kg of the compound.
265. The method of embodiment 260, wherein the amount of the one or more food products administered to the subject per day contains 0.01 mg/kg to 3 mg/kg of the compound.
266. The method of embodiment 260, wherein the amount of the one or more food products administered to the subject per day contains 0.01 mg/kg to 1 mg/kg of the compound.
267. The method of embodiment 260, wherein the amount of the one or more food products administered to the subject per day contains 0.02 mg/kg to 5 mg/kg of the compound.
268. The method of embodiment 260, wherein the amount of the one or more food products administered to the subject per day contains 0.02 mg/kg to 2 mg/kg of the compound.
269. The method of embodiment 260, wherein the amount of the one or more food products administered to the subject per day contains 0.02 mg/kg to 1 mg/kg of the compound.
270. The method of embodiment 260, wherein the amount of the one or more food products administered to the subject per day contains 0.1 mg to 50 g of the compound.
271. The method of embodiment 260, wherein the amount of the one or more food products administered to the subject per day contains 0.1 mg to 10 g of the compound.
272. The method of embodiment 260, wherein the amount of the one or more food products administered to the subject per day contains 0.1 mg to 3 g of the compound.
273. The method of embodiment 260, wherein the amount of the one or more food products administered to the subject per day contains 0.1 mg to 100 mg of the compound.
274. The method of embodiment 260, wherein the amount of the one or more food products administered to the subject per day contains 0.1 mg to 1 mg of the compound.
275. The method of embodiment 260, wherein the amount of the one or more food products administered to the subject per day contains 0.3 mg to 3 g of the compound.
276. The method of embodiment 260, wherein the amount of the one or more food products administered to the subject per day contains 0.3 mg to 100 mg of the compound.
277. A compound according to any one of embodiments 1 to 213 for use as a medicament.
278. A compound according to any one of embodiments 1 to 213 for use in a method for the treatment of a mood disorder.
279. The compound for use according to embodiment 278, wherein the mood disorder comprises depression, bipolar disorder, or adjustment disorder.
280. The compound for use according to embodiment 279, wherein the mood disorder comprises depression.
281. The compound for use according to embodiment 279, wherein the mood disorder comprises bipolar disorder.
282. The compound for use according to embodiment 279, wherein the mood disorder comprises adjustment disorder.
283. A compound according to any one of embodiments 1 to 213 for use in a method for the treatment of an anxiety disorder.
284. A compound according to any one of embodiments 1 to 213 for use in a method for the treatment of a disorder of diminished motivation.
285. The compound for use according to embodiment 284, wherein the disorder of diminished motivation comprises apathy, abulia, or akinetic mutism.
286. The compound for use according to embodiment 285, wherein the disorder of diminished motivation comprises apathy.
287. The compound for use according to embodiment 285, wherein the disorder of diminished motivation comprises abulia.
288. The compound for use according to embodiment 285, wherein the disorder of diminished motivation comprises akinetic mutism.
289. The compound for use according to any one of embodiments 278 to 288, wherein the method comprises orally administering an effective amount of the compound to a subject.
290. The compound for use according to embodiment 289, wherein the method comprises administering 0.005 mg/kg to 500 mg/kg of the compound to the subject per day.
291. The compound for use according to embodiment 289, wherein the method comprises administering 0.005 mg/kg to 100 mg/kg of the compound to the subject per day.
292. The compound for use according to embodiment 289, wherein the method comprises administering 0.005 mg/kg to 30 mg/kg of the compound to the subject per day.
293. The compound for use according to embodiment 289, wherein the method comprises administering 0.005 mg/kg to 1 mg/kg of the compound to the subject per day.
294. The compound for use according to embodiment 289, wherein the method comprises administering 0.01 mg/kg to 30 mg/kg of the compound to the subject per day.
   295. The compound for use according to embodiment 289, wherein the method comprises administering 0.01 mg/kg to 3 mg/kg of the compound to the subject per day.
296. The compound for use according to embodiment 289, wherein the method comprises administering 0.01 mg/kg to 1 mg/kg of the compound to the subject per day.
297. The compound for use according to embodiment 289, wherein the method comprises administering 0.02 mg/kg to 5 mg/kg of the compound to the subject per day.
298. The compound for use according to embodiment 289, wherein the method comprises administering 0.02 mg/kg to 2 mg/kg of the compound to the subject per day.
299. The compound for use according to embodiment 289, wherein the method comprises administering 0.02 mg/kg to 1 mg/kg of the compound to the subject per day.
300. The compound for use according to embodiment 289, wherein the method comprises administering 0.1 mg to 50 g of the compound to the subject per day.
301. The compound for use according to embodiment 289, wherein the method comprises administering 0.1 mg to 10 g of the compound to the subject per day.
302. The compound for use according to embodiment 289, wherein the method comprises administering 0.1 mg to 3 g of the compound to the subject per day.
303. The compound for use according to embodiment 289, wherein the method comprises administering 0.1 mg to 100 mg of the compound to the subject per day.
304. The compound for use according to embodiment 289, wherein the method comprises administering 0.1 mg to 1 mg of the compound to the subject per day.
305. The compound for use according to embodiment 289, wherein the method comprises administering 0.3 mg to 3 g of the compound to the subject per day.
306. The compound for use according to embodiment 289, wherein the method comprises administering 0.3 mg to 100 mg of the compound to the subject per day.
307. A pharmaceutical composition according to embodiment 214 for use in a method for the treatment of a mood disorder.
308. The pharmaceutical composition for use according to embodiment 307, wherein the mood disorder comprises depression, bipolar disorder, or adjustment disorder.
309. The pharmaceutical composition for use according to embodiment 307, wherein the mood disorder comprises depression.
310. The pharmaceutical composition for use according to embodiment 307, wherein the mood disorder comprises bipolar disorder.
311. The pharmaceutical composition for use according to embodiment 307, wherein the mood disorder comprises adjustment disorder.
312. A pharmaceutical composition according to embodiment 214 for use in a method for the treatment of an anxiety disorder.
313. A pharmaceutical composition according to embodiment 214 for use in a method for the treatment of a disorder of diminished motivation.
314. The pharmaceutical composition for use according to embodiment 313, wherein the disorder of diminished motivation comprises apathy, abulia, or akinetic mutism.
315. The pharmaceutical composition for use according to embodiment 313, wherein the disorder of diminished motivation comprises apathy.
316. The pharmaceutical composition for use according to embodiment 313, wherein the disorder of diminished motivation comprises abulia.
317. The pharmaceutical composition for use according to embodiment 313, wherein the disorder of diminished motivation comprises akinetic mutism.
318. The pharmaceutical composition for use according to any one of embodiments 307 to 317, wherein the method comprises orally administering an effect amount of the pharmaceutical composition to a subject.
319. The pharmaceutical composition for use according to embodiment 318, wherein the method comprises administering an amount of the pharmaceutical composition to the subject per day that contains 0.005 mg/kg to 500 mg/kg of the compound.
320. The pharmaceutical composition for use according to embodiment 318, wherein the method comprises administering an amount of the pharmaceutical composition to the subject per day that contains 0.005 mg/kg to 100 mg/kg of the compound.
321. The pharmaceutical composition for use according to embodiment 318, wherein the method comprises administering an amount of the pharmaceutical composition to the subject per day that contains 0.005 mg/kg to 30 mg/kg of the compound.
322. The pharmaceutical composition for use according to embodiment 318, wherein the method comprises administering an amount of the pharmaceutical composition to the subject per day that contains 0.005 mg/kg to 1 mg/kg of the compound.
323. The pharmaceutical composition for use according to embodiment 318, wherein the method comprises administering an amount of the pharmaceutical composition to the subject per day that contains 0.01 mg/kg to 30 mg/kg of the compound.
324. The pharmaceutical composition for use according to embodiment 318, wherein the method comprises administering an amount of the pharmaceutical composition to the subject per day that contains 0.01 mg/kg to 3 mg/kg of the compound.
325. The pharmaceutical composition for use according to embodiment 318, wherein the method comprises administering an amount of the pharmaceutical composition to the subject per day that contains 0.01 mg/kg to 1 mg/kg of the compound.
326. The pharmaceutical composition for use according to embodiment 318, wherein the method comprises administering an amount of the pharmaceutical composition to the subject per day that contains 0.02 mg/kg to 5 mg/kg of the compound.
327. The pharmaceutical composition for use according to embodiment 318, wherein the method comprises administering an amount of the pharmaceutical composition to the subject per day that contains 0.02 mg/kg to 2 mg/kg of the compound.
328. The pharmaceutical composition for use according to embodiment 318, wherein the method comprises administering an amount of the pharmaceutical composition to the subject per day that contains 0.02 mg/kg to 1 mg/kg of the compound.
329. The pharmaceutical composition for use according to embodiment 318, wherein the method comprises administering an amount of the pharmaceutical composition to the subject per day that contains 0.1 mg to 50 g of the compound.
330. The pharmaceutical composition for use according to embodiment 318, wherein the method comprises administering an amount of the pharmaceutical composition to the subject per day that contains 0.1 mg to 10 g of the compound.
331. The pharmaceutical composition for use according to embodiment 318, wherein the method comprises administering an amount of the pharmaceutical composition to the subject per day that contains 0.1 mg to 3 g of the compound.
332. The pharmaceutical composition for use according to embodiment 318, wherein the method comprises administering an amount of the pharmaceutical composition to the subject per day that contains 0.1 mg to 100 mg of the compound.
333. The pharmaceutical composition for use according to embodiment 318, wherein the method comprises administering an amount of the pharmaceutical composition to the subject per day that contains 0.1 mg to 1 mg of the compound.
334. The pharmaceutical composition for use according to embodiment 318, wherein the method comprises administering an amount of the pharmaceutical composition to the subject per day that contains 0.3 mg to 3 g of the compound.
335. The pharmaceutical composition for use according to embodiment 318, wherein the method comprises administering an amount of the pharmaceutical composition to the subject per day that contains 0.3 mg to 100 mg of the compound.
336. A food product according to any one of embodiments 215 to 217 for use in a method for the treatment of or prevention of a mood disorder.
337. The food product for use according to embodiment 336, wherein the mood disorder comprises depression, bipolar disorder, or adjustment disorder.
338. The food product for use according to embodiment 337, wherein the mood disorder comprises depression.
339. The food product for use according to embodiment 337, wherein the mood disorder comprises bipolar disorder.
340. The food product for use according to embodiment 337, wherein the mood disorder comprises adjustment disorder.
341. A food product according to any one of embodiments 215 to 217 for use in a method for the treatment of or prevention of an anxiety disorder.
342. A food product for use according to any one of embodiments 215 to 217 for use in a method for the treatment of or prevention of a disorder of diminished motivation.
343. The food product for use according to embodiment 342, wherein the disorder of diminished motivation comprises apathy, abulia, or akinetic mutism.
344. The food product for use according to embodiment 343, wherein the disorder of diminished motivation comprises apathy.
345. The food product for use according to embodiment 343, wherein the disorder of diminished motivation comprises abulia.
346. The food product for use according to embodiment 343, wherein the disorder of diminished motivation comprises akinetic mutism.
347. The food product for use according to any one of embodiments 336 to 346, wherein the method comprises administering an amount of one or more food products according to any one of embodiments 215 to 217 to a subject, and wherein the amount of the one or more food products administered to the subject per day contains 0.005 mg/kg to 500 mg/kg of the compound.
348. The food product for use according to any one of embodiments 336 to 346, wherein the method comprises administering an amount of one or more food products according to any one of embodiments 215 to 217 to a subject, and wherein the amount of the one or more food products administered to the subject per day contains 0.005 mg/kg to 100 mg/kg of the compound.
349. The food product for use according to any one of embodiments 336 to 346, wherein the method comprises administering an amount of one or more food products according to any one of embodiments 215 to 217 to a subject, and wherein the amount of the one or more food products administered to the subject per day contains 0.005 mg/kg to 30 mg/kg of the compound.
350. The food product for use according to any one of embodiments 336 to 346, wherein the method comprises administering an amount of one or more food products according to any one of embodiments 215 to 217 to a subject, and wherein the amount of the one or more food products administered to the subject per day contains 0.005 mg/kg to 1 mg/kg of the compound.
351. The food product for use according to any one of embodiments 336 to 346, wherein the method comprises administering an amount of one or more food products according to any one of embodiments 215 to 217 to a subject, and wherein the amount of the one or more food products administered to the subject per day contains 0.01 mg/kg to 30 mg/kg of the compound.
352. The food product for use according to any one of embodiments 336 to 346, wherein the method comprises administering an amount of one or more food products according to any one of embodiments 215 to 217 to a subject, and wherein the amount of the one or more food products administered to the subject per day contains 0.01 mg/kg to 3 mg/kg of the compound.
353. The food product for use according to any one of embodiments 336 to 346, wherein the method comprises administering an amount of one or more food products according to any one of embodiments 215 to 217 to a subject, and wherein the amount of the one or more food products administered to the subject per day contains 0.01 mg/kg to 1 mg/kg of the compound.
354. The food product for use according to any one of embodiments 336 to 346, wherein the method comprises administering an amount of one or more food products according to any one of embodiments 215 to 217 to a subject, and wherein the amount of the one or more food products administered to the subject per day contains 0.02 mg/kg to 5 mg/kg of the compound.
355. The food product for use according to any one of embodiments 336 to 346, wherein the method comprises administering an amount of one or more food products according to any one of embodiments 215 to 217 to a subject, and wherein the amount of the one or more food products administered to the subject per day contains 0.02 mg/kg to 2 mg/kg of the compound.
356. The food product for use according to any one of embodiments 336 to 346, wherein the method comprises administering an amount of one or more food products according to any one of embodiments 215 to 217 to a subject, and wherein the amount of the one or more food products administered to the subject per day contains 0.02 mg/kg to 1 mg/kg of the compound.
357. The food product for use according to any one of embodiments 336 to 346, wherein the method comprises administering an amount of one or more food products according to any one of embodiments 215 to 217 to a subject, and wherein the amount of the one or more food products administered to the subject per day contains 0.1 mg to 50 g of the compound.
358. The food product for use according to any one of embodiments 336 to 346, wherein the method comprises administering an amount of one or more food products according to any one of embodiments 215 to 217 to a subject, and wherein the amount of the one or more food products administered to the subject per day contains 0.1 mg to 10 g of the compound.
359. The food product for use according to any one of embodiments 336 to 346, wherein the method comprises administering an amount of one or more food products according to any one of embodiments 215 to 217 to a subject, and wherein the amount of the one or more food products administered to the subject per day contains 0.1 mg to 3 g of the compound.
360. The food product for use according to any one of embodiments 336 to 346, wherein the method comprises administering an amount of one or more food products according to any one of embodiments 215 to 217 to a subject, and wherein the amount of the one or more food products administered to the subject per day contains 0.1 mg to 100 mg of the compound.
361. The food product for use according to any one of embodiments 336 to 346, wherein the method comprises administering an amount of one or more food products according to any one of embodiments 215 to 217 to a subject, and wherein the amount of the one or more food products administered to the subject per day contains 0.1 mg to 1 mg of the compound.
362. The food product for use according to any one of embodiments 336 to 346, wherein the method comprises administering an amount of one or more food products according to any one of embodiments 215 to 217 to a subject, and wherein the amount of the one or more food products administered to the subject per day contains 0.3 mg to 3 g of the compound.
363. The food product for use according to any one of embodiments 336 to 346, wherein the method comprises administering an amount of one or more food products according to any one of embodiments 215 to 217 to a subject, and wherein the amount of the one or more food products administered to the subject per day contains 0.3 mg to 100 mg of the compound.
364. A composition comprising RubisCo protein treated with pepsin or pepsin and pancreatin for use in a method for the treatment of an anxiety disorder, a mood disorder, or a disorder of diminished motivation.
365. The composition for use according to embodiment 364, wherein the RubisCo protein is spinach RubisCo.
366. The composition for use according to embodiment 364 or embodiment 365, for use in a method for the treatment of an anxiety disorder.
367. The composition for use according to embodiment 364 or embodiment 365, for use in a method for the treatment of a mood disorder.
368. The composition for use according to embodiment 367, wherein the mood disorder comprises depression, bipolar disorder, or adjustment disorder.
369. The composition for use according to embodiment 364 or embodiment 365, for use in a method for the treatment of a disorder of diminished motivation.
370. The composition for use according to embodiment 369, wherein the disorder of diminished motivation comprises apathy, abulia, or akinetic mutism.

### 8. Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

### 9. Sequence Listing

TABLE 2

| Amino Acid Sequence | Comments | SEQ ID NO: |
|---|---|---|
| SYLPPLTT | | 1 |
| YHIEPV | | 2 |
| YLLVK | | 3 |
| SYLPPLT | | 4 |
| FLLVK | | 5 |
| WLLVK | | 6 |
| YLL | | 7 |
| X₁LX₂X₃VK | X₁ is a hydrophobic amino acid; | 8 |
| | X₂ and X₃ are each independently | |
| | selected from any amino acid | |
| LVK | | 9 |
| YLLV | | 10 |
| LLVK | | 11 |
| YLVK | | 12 |
| YLLVR | | 13 |
| YLPPL | | 14 |
| X₁LX₂ | X₁ is a hydrophobic amino acid; | 15 |
| | X₂ is selected from any amino acid | |
| X₃VK | X₃ is selected from any amino acid | 16 |
| X₁LX₂X₃ | X₁ is a hydrophobic amino acid; | 17 |
| | X₂ and X₃ are each independently | |
| | selected from any amino acid | |
| X₂X₃VK | X₂ and X₃ are each independently | 18 |
| | selected from any amino acid | |
| X₁LX₂X₃V | X₁ is a hydrophobic amino acid; | 19 |
| | X₂ and X₃ are each independently | |
| | selected from any amino acid | |
| SYLPPL | | 20 |
| NYLLVKG | | 21 |
| YLLAVK | | 22 |
| YLLNNK | | 23 |
| SYLPP | | 24 |
| XLLVK | X is an aromatic amino acid | 25 |
| YETLSYLPPLTTDQLAR | | 26 |
| LX₂X₃VK | X₂ and X₃ are each independently | 27 |
| | selected from any amino acid | |
| YKGRCYHIEPVAGEEN | | 28 |
| X₁LX₂X₃K | X₁ is a hydrophobic amino acid; | 29 |
| | X₂ and X₃ are each independently | |
| | selected from any amino acid | |
| X₄LX₅ | X₄ is Y, F, W, or H; X₅ is Y, F, | 30 |
| | W, Q, or L | |

TABLE 3

| | | |
|---|---|---|
| X₄LX₅EIAR | X₄ is Y, F, W, or H; X₅ is | 31 |
| | Y, F, W, Q, or L | |
| VYLPR | | 32 |
| YLPR | | 33 |
| VLQRF | | 34 |

1. A compound that is a peptide or a salt thereof, wherein the peptide:
(a) is 3 to 20 amino acids in length;
(b) has an amino acid sequence comprising at least 3 consecutive amino acids from the N-terminal or C-terminal end of the amino acid sequence X₁LX₂X₃VK (SEQ ID NO:8), wherein
(i) X₁ is a hydrophobic amino acid,
(ii) X₂ and X₃ are each independently selected from any amino acid;
(c) has a hydrophobic N-terminal amino acid; and
(d) does not consist of the amino acid sequence YLLVR (SEQ ID NO:13), X₄LX₅ (SEQ ID NO:30), X₄LX₅EIAR (SEQ ID NO:31), VYLPR (SEQ ID NO:32), YLPR (SEQ ID NO:33), or VLQRF (SEQ ID NO:34), where X₄ is Y, F, W, or H and X₅ is Y, F, W, Q, or L.

2. The compound of claim 1, wherein X₁ is an aromatic amino acid.

3. The compound of claim 1, wherein X₁ is Y, F, W, A, I, L, V, or M.

4. The compound of claim 1, wherein X₁ is Y, F, or W.

5. The compound of claim 1, wherein X₂ and/or X₃ is L, I, V, or A.

6. The compound of claim 1, wherein the peptide comprises the amino acid sequence:
(a) X₁LX₂ (SEQ ID NO:15), optionally wherein X₁ is the N-terminal amino acid of the peptide;
(b) X₃VK (SEQ ID NO:16);
(c) YLL (SEQ ID NO:7); or
(d) LVK (SEQ ID NO:9).

7. The compound of claim 1,
wherein the peptide comprises the amino acid sequence:
(a) X₁LX₂X₃ (SEQ ID NO:17), optionally wherein:
X₁ is the N-terminal amino acid of the peptide; and/or
the peptide comprises the amino acid sequence X₁LX₂X₃K (SEQ ID NO:29);
(b) X₂X₃VK (SEQ ID NO:18);
(c) YLLV (SEQ ID NO:10);
(d) LLVK (SEQ ID NO:11); or
(e) YLVK (SEQ ID NO:12).

8. The compound of claim 1,
wherein the peptide comprises the amino acid sequence:
(a) X₁LX₂X₃V (SEQ ID NO:19), optionally wherein X₁ is the N-terminal amino acid of the peptide;
(b) LX₂X₃VK (SEQ ID NO:27);
(c) YLLVK (SEQ ID NO:3);
(d) FLLVK (SEQ ID NO:5);
(e) WLLVK (SEQ ID NO:6); or
(f) YLLVR (SEQ ID NO:13).

9. The compound of claim 1, wherein the peptide comprises the amino acid sequence:
(a) X₁LX₂X₃VK (SEQ ID NO:8), optionally wherein X₁ is the N-terminal amino acid of the peptide;
(b) YLLAVK (SEQ ID NO:22); or
(c) YLLNNK (SEQ ID NO:23).

10. The compound of claim 1, wherein the peptide is 3 to 15 amino acids in length or 3 to 10 amino acids in length.

11. (canceled)

12. The compound of claim 1, wherein the peptide is 5 to 20 amino acids in length, 5 to 15 amino acids in length, or 5 to 10 amino acids in length.

13-17. (canceled)

18. A compound that is a peptide or a salt thereof, wherein the peptide:
(a) is 5 to 20 amino acids in length; and
(b) comprises the amino acid sequence SYLPP (SEQ ID NO:24) at the N-terminal end of the peptide.

19-26. (canceled)

27. A compound that is a peptide or a salt thereof, wherein the peptide:
(a) is 6 to 20 amino acids in length; and
(b) comprises the amino acid sequence YHIEPV (SEQ ID NO:2).

28-32. (canceled)

33. A compound that is a conjugate or a salt thereof, the conjugate comprising:
(a) a peptide moiety that:
(i) is 3 to 20 amino acids in length;
(ii) has an amino acid sequence comprising at least 3 consecutive amino acids from the N-terminal or C-terminal end of the amino acid sequence X₁LX₂X₃VK (SEQ ID NO:8), wherein
X₁ is a hydrophobic amino acid,
X₂ and X₃ are each independently selected from any amino acid; and
(iii) optionally, has a hydrophobic N-terminal amino acid;
attached to
(b) one or more conjugate moieties.

34-39. (canceled)

40. The compound of claim 33, wherein the peptide moiety comprises the amino acid sequence:
(a) X₁LX₂X₃V (SEQ ID NO:19), optionally wherein X₁ is the N-terminal amino acid of the peptide moiety;
(b) LX₂X₃VK (SEQ ID NO:27);
(c) YLLVK (SEQ ID NO:3);
(d) FLLVK (SEQ ID NO:5);
(e) WLLVK (SEQ ID NO:6); or
(f) YLLVR (SEQ ID NO:13).

41. (canceled)

42. A compound that is a conjugate or a salt thereof, the conjugate comprising a peptide moiety consisting of the peptide of claim 18 and one or more conjugate moieties.

43-44. (canceled)

45. A pharmaceutical composition comprising the compound of claim 1 and one or more pharmaceutically acceptable carriers, diluents and/or excipients.

46. A food product comprising as an additive the compound of claim 1.

47. A compound according to claim 1 for use in a method for the treatment of an anxiety disorder, a mood disorder, or a disorder of diminished motivation.

48-52. (canceled)

53. A pharmaceutical composition according to claim 45 for use in a method for the treatment of an anxiety disorder, a mood disorder, or a disorder of diminished motivation.

54-58. (canceled)

59. A food product according to claim 46 for use in a method for the treatment of an anxiety disorder, a mood disorder, or a disorder of diminished motivation.

60-64. (canceled)
